SEQUENCE LISTING (45 sequences)

SEQ ID NO: 1 - protein, 4 aa, Artificial Sequence
  Synthetic immune-stimulating peptide
YGGF

SEQ ID NO: 2 - protein, 4 aa, Artificial Sequence
  Synthetic immune-stimulating peptide
YGGW

SEQ ID NO: 3 - protein, 8 aa, Artificial Sequence
  Synthetic immune-stimulating peptide
YGGFGGFG

SEQ ID NO: 4 - protein, 8 aa, Artificial Sequence
  Synthetic immune-stimulating peptide
  SITE (7)..(7): Xaa is Dip (3,3-diphenylalanine)
YGGFGGXG

SEQ ID NO: 5 - protein, 8 aa, Artificial Sequence
  Synthetic immune-stimulating peptide
  SITE (7)..(7): Xaa is Bip (biphenylalanine)
YGGFGGXG

SEQ ID NO: 6 - protein, 8 aa, Artificial Sequence
  Synthetic immune-stimulating peptide
  SITE (7)..(7): Xaa is Ath (9-anthracenylalanine)
YGGFGGXG

SEQ ID NO: 7 - protein, 8 aa, Artificial Sequence
  Synthetic immune-stimulating peptide
  SITE (7)..(7): Xaa is Dap (2,3-diaminopropionic acid)
YGGFGGXG

SEQ ID NO: 8 - protein, 8 aa, Artificial Sequence
  Synthetic immune-stimulating peptide
  SITE (7)..(7): Xaa is Dab (2,4-diaminobutanoic acid)
YGGFGGXG

SEQ ID NO: 9 - protein, 8 aa, Artificial Sequence
  Synthetic immune-stimulating peptide
  SITE (7)..(7): Xaa is Nal (1- naphthylalanine)
YGGFGGXG

SEQ ID NO: 10 - protein, 8 aa, Artificial Sequence
  Synthetic immune-stimulating peptide
YGGWGGFG

SEQ ID NO: 11 - protein, 8 aa, Artificial Sequence
  Synthetic immune-stimulating peptide
YGGWGGWG

SEQ ID NO: 12 - protein, 5 aa, Artificial Sequence
  Synthetic immune-stimulating peptide
YGGFM

SEQ ID NO: 13 - protein, 5 aa, Artificial Sequence
  Synthetic immune-stimulating peptide
  SITE (4)..(4): Xaa is Dip (3,3-diphenylalanine)
YGGXM

SEQ ID NO: 14 - protein, 5 aa, Artificial Sequence
  Synthetic immune-stimulating peptide
  SITE (4)..(4): Xaa is Bip (biphenylalanine)
YGGXM

SEQ ID NO: 15 - protein, 5 aa, Artificial Sequence
  Synthetic immune-stimulating peptide
  SITE (4)..(4): Xaa is Ath (9-anthracenylalanine)
YGGXM

SEQ ID NO: 16 - protein, 5 aa, Artificial Sequence
  Synthetic immune-stimulating peptide
  SITE (4)..(4): Xaa is Dap (2,3-diaminopropionic acid)
YGGXM

SEQ ID NO: 17 - protein, 5 aa, Artificial Sequence
  Synthetic immune-stimulating peptide
  SITE (4)..(4): Xaa is Dab (2,4-diaminobutanoic acid)
YGGXM

SEQ ID NO: 18 - protein, 5 aa, Artificial Sequence
  Synthetic immune-stimulating peptide
  SITE (4)..(4): Xaa is Nal (1- naphthylalanine)
YGGXM

SEQ ID NO: 19 - protein, 9 aa, Artificial Sequence
  Synthetic immune-stimulating peptide
YGGWMYGAF

SEQ ID NO: 20 - protein, 4 aa, Artificial Sequence
  Synthetic immune-stimulating peptide
YGAW

SEQ ID NO: 21 - protein, 5 aa, Artificial Sequence
  Synthetic immune-stimulating peptide
  SITE (3)..(3): Xaa is Aib (Alpha-aminoisobutyric acid)
YGXFM

SEQ ID NO: 22 - protein, 5 aa, Artificial Sequence
  Synthetic immune-stimulating peptide
  SITE (4)..(4): Xaa is Dip (3,3-diphenylalanine)
YGAXM

SEQ ID NO: 23 - protein, 5 aa, Artificial Sequence
  Synthetic immune-stimulating peptide
  SITE (4)..(4): Xaa is Bip (biphenylalanine)
YGAXM

SEQ ID NO: 24 - protein, 5 aa, Artificial Sequence
  Synthetic immune-stimulating peptide
  SITE (4)..(4): Xaa is Ath (9-anthracenylalanine)
YGAXM

SEQ ID NO: 25 - protein, 5 aa, Artificial Sequence
  Synthetic immune-stimulating peptide
  SITE (4)..(4): Xaa is Dap (2,3-diaminopropionic acid)
YGAXM

SEQ ID NO: 26 - protein, 5 aa, Artificial Sequence
  Synthetic immune-stimulating peptide
  SITE (4)..(4): Xaa is Dab (2,4-diaminobutanoic acid)
YGAXM

SEQ ID NO: 27 - protein, 5 aa, Artificial Sequence
  Synthetic immune-stimulating peptide
  SITE (4)..(4): Xaa is Nal (1- naphthylalanine)
YGAXM

SEQ ID NO: 28 - protein, 5 aa, Artificial Sequence
  Synthetic immune-stimulating peptide
YGGWM

SEQ ID NO: 29 - protein, 4 aa, Artificial Sequence
  Synthetic immune-stimulating peptide
YGAF

SEQ ID NO: 30 - protein, 4 aa, Artificial Sequence
  Synthetic immune-stimulating peptide
YAGF

SEQ ID NO: 31 - protein, 4 aa, Artificial Sequence
  Synthetic immune-stimulating peptide
YAGW

SEQ ID NO: 32 - protein, 5 aa, Artificial Sequence
  Synthetic immune-stimulating peptide
  SITE (2)..(2): Xaa is Aib (Alpha-aminoisobutyric acid)
YXGFM

SEQ ID NO: 33 - protein, 9 aa, Artificial Sequence
  Synthetic immune-stimulating peptide
ACYGGGACG

SEQ ID NO: 34 - protein, 9 aa, Artificial Sequence
  Synthetic immune-stimulating peptide
ACYGGFACG

SEQ ID NO: 35 - protein, 9 aa, Artificial Sequence
  Synthetic immune-stimulating peptide
  SITE (6)..(6): Xaa is Bip (biphenylalanine)
ACYGGXACG

SEQ ID NO: 36 - protein, 9 aa, Artificial Sequence
  Synthetic immune-stimulating peptide
  SITE (6)..(6): Xaa is Dip (3,3-diphenylalanine)
ACYGGXACG

SEQ ID NO: 37 - protein, 9 aa, Artificial Sequence
  Synthetic immune-stimulating peptide
  SITE (6)..(6): Xaa is Ath (9-anthracenylalanine)
ACYGGXACG

SEQ ID NO: 38 - protein, 9 aa, Artificial Sequence
  Synthetic immune-stimulating peptide
  SITE (6)..(6): Xaa is Dab (2,4-diaminobutanoic acid)
ACYGGXACG

SEQ ID NO: 39 - protein, 9 aa, Artificial Sequence
  Synthetic immune-stimulating peptide
  SITE (6)..(6): Xaa is Dap (2,3-diaminopropionic acid)
ACYGGXACG

SEQ ID NO: 40 - protein, 9 aa, Artificial Sequence
  Synthetic immune-stimulating peptide
  SITE (6)..(6): Xaa is Nal (1- naphthylalanine)
ACYGGXACG

SEQ ID NO: 41 - protein, 9 aa, Artificial Sequence
  Synthetic immune-stimulating peptide
ACYGGGWCG

SEQ ID NO: 42 - protein, 5 aa, Artificial Sequence
  Synthetic immune-stimulating peptide
YGGFL

SEQ ID NO: 43 - protein, 4 aa, Artificial Sequence
  Synthetic immune-stimulating peptide
YGYG

SEQ ID NO: 44 - protein, 6 aa, Artificial Sequence
  Synthetic immune-stimulating peptide
YGYGYG

SEQ ID NO: 45 - protein, 8 aa, Artificial Sequence
  Synthetic immune-stimulating peptide
YGYGYGYG

(19) United States
(12) Patent Application Publication
(10) Pub. No.: US 2019/0241660 A1
(43) Pub. Date: Aug. 8, 2019

(54) IMMUNE-STIMULATING PEPTIDES AND CHECKPOINT INHIBITORS, AND USES THEREOF FOR TREATING CANCER

(71) Applicant: INTENDET SERVICES, LLC, Chestnut Hill, MA (US)

(72) Inventors: Brett P. GIROIR, College Station, TX (US); Clifford H. KERN, III, Metairie, LA (US); Carola LEUSCHNER, Houston, TX (US)

(21) Appl. No.: 16/209,143

(22) Filed: Dec. 4, 2018

Related U.S. Application Data
(60) Provisional application No. 62/594,829, filed on Dec. 5, 2017.

(51) Int. Cl. C07K 16/28; C07K 5/06; A61K 35/17; A61P 35/00; A61P 31/00; A61K 39/00; C12N 5/0783

(52) U.S. Cl. CPC C07K 16/2818; C07K 5/06; A61K 35/17; A61P 35/00; A61K 2039/5158; A61P 31/00; A61K 39/0011; C12N 5/0636; A61K 2039/5154; C07K 16/2827

(57) ABSTRACT

Presented herein are immune-stimulating peptides and uses thereof for treating cancer. The immune-stimulating peptides can be used alone, or in combination with checkpoint inhibitors and other anti-cancer therapies. The immune-stimulating peptides can also be used for expanding T-cells in vitro or ex vivo, which expanded T-cells can then be used for adoptive cell therapy.

### RELATED PATENT APPLICATION

This patent application claims the benefit of Provisional Patent Application No. 62/594,829 filed on Dec. 5, 2017 entitled “IMMUNE-STIMULATING PEPTIDES AND CHECKPOINT INHIBITORS, AND USES THEREOF FOR TREATING CANCER”, naming Brett P. Giroir, Clifford H. Kern III, and Carola Leuschner as inventors, and designated by attorney docket no. 038524-0454941. The entire content of the foregoing patent application is incorporated herein by reference, including all text, tables and drawings.

### FIELD OF THE INVENTION

Embodiments relate to immune-stimulating peptides and checkpoint inhibitors, and uses thereof in combination therapy for treating cancer. Also, certain embodiments relate to the use of immune-stimulating peptides in combination with other anti-cancer therapies.

### INTRODUCTION

Several immunotherapies have been shown to reduce tumor progression and enhance survival in humans. Among these are antibodies directed to immune inhibitory molecules often referred to as checkpoint proteins.

Programmed Death protein 1 (PD-1) and Cytotoxic T-Lymphocyte Associated Protein 4 (CTLA-4) are examples of checkpoint protein that are expressed on immune cells such as T-cells. These checkpoint proteins normally act as a type of “off switch” that helps keep a T cell from activating and attacking other cells in the body. For example, the binding of PD-1 to its ligand PD-L1 often results in an inhibitory signal within a T-cell that prevents activation of the T-cell. Some cancer cells express large amounts of PD-L1, which helps them evade immune attack from T-cells. CTLA-4 acts in a similar manner and induces an inhibitory signal in a T-cell when it engages its cognate B7 family ligands, e.g., CD80 and CD86.

Checkpoint inhibitors are often antibodies or small compounds that bind to checkpoint proteins such as PD-1 and CTLA-4, or to their cognate ligands, and block signaling through the checkpoint proteins by their natural ligands. Accordingly, checkpoint inhibitors allow activation of T-cells so that they can engage and kill cancer cells. Checkpoint inhibitors have shown some success in the clinic for the treatment of cancer.

Presented herein are novel small peptides that can stimulate an immune response by modulating immune cells such as T-cell subpopulations (e.g., CD8+ and CD4+ T cells, and regulatory T-cells), natural killer cells, macrophages, dendritic cells and enhance the anti-cancer effects of checkpoint inhibitors.

### SUMMARY

In some aspects, presented herein is a composition comprising an (i) immune-stimulating peptide and (ii) a checkpoint inhibitor. In certain aspects, presented herein is a method of treating a cancer in a subject in need thereof comprising administering to the subject a therapeutically effective amount of an immune-stimulating peptide and a therapeutically effective amount of a checkpoint inhibitor. In certain aspects, presented herein is a method of treating a cancer in a subject in need thereof comprising administering to the subject a composition comprising a therapeutically effective amount of an immune-stimulating peptide and a therapeutically effective amount of a checkpoint inhibitor.

In certain embodiments, an immune-stimulating peptide comprises the amino acid sequence Tyr-Gly. In certain embodiments, the amino acid sequence Tyr-Gly is located on the N-terminal end or C-terminal end of the immune-stimulating peptide. In certain embodiments, the immune-stimulating peptide consists of the amino acid sequence Tyr-Gly.

In certain embodiments, a checkpoint inhibitor comprises an antibody or compound that inhibits, blocks, ameliorates, or suppresses signal transduction through Cytotoxic T-lymphocyte protein 4 (CTLA-4) or Programmed Cell Death protein 1 (PD-1). In certain embodiments, a checkpoint inhibitor comprises an antibody or compound that inhibits, blocks, ameliorates, or suppresses activation of a T-cell, wherein the activation of the T-cell is mediated by signal transduction through CTLA-4 or PD-1. In certain embodiments, a checkpoint inhibitor is an antibody or compound that inhibits, blocks, ameliorates, or suppresses binding of CTLA-4 to CD86 (B7-2), CD80 (B7-1) or B7-Related protein (ICOS Ligand, CD275). In some embodiments, a checkpoint inhibitor is an antibody that binds specifically to CTLA-4 on the surface of a T-cell and inhibits, blocks, ameliorates, or suppresses activation of the T-cell. In certain embodiments, a checkpoint inhibitor is an antibody that specifically binds to PD-1, PD-L1 or PD-L2. In certain embodiments, a checkpoint inhibitor is an antibody or compound that inhibits, blocks, ameliorates, or suppresses binding of PD-1 to PD-L1 or PD-L2. In certain embodiments, a checkpoint inhibitor is a monoclonal antibody, or binding fragment thereof. In some embodiments, a checkpoint inhibitor is a chimeric, humanized or human monoclonal antibody.

Certain aspects of the technology are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 shows a graphical representation of tumor growth in mice treated with a vehicle control (Vehicle, closed circles w/solid line), anti-CTLA-4 antibody (anti-CTLA4, open squares, w/solid line), INT-151 (INT-151, open upward triangles, w/dashed line) or INT-151 and anti-CTLA-4 antibody (Combination, inverted open triangles w/solid line). Tumor growth was plotted as tumor volume (Y-axis, mm³) over time (X-axis, days). The graphs display median values over time.

FIG. 2 shows a graphical representation of a change in body-weight (Y-axis, grams (g)) over time (X-axis, days) of mice implanted with tumor cells on day 1 and treated with a vehicle control (Vehicle, closed circles w/solid line), anti-CTLA-4 antibody (anti-CTLA4, open squares, w/solid line), INT-151 (INT-151, open upward triangles, w/dashed line) or INT-151 and anti-CTLA-4 antibody (Combination, inverted open triangles w/solid line). The graph displays median values over time.

FIG. 3 shows a survival curve of mice implanted with tumor cells on day 1 and treated with a vehicle control (Vehicle, dark solid line), anti-CTLA-4 antibody (grey solid line), INT-151 (dotted line) or INT-151 and anti-CTLA-4 antibody (Combination, dashed line). The X-axis represents time after implantation (days) and the Y-axis represents the percentage of mice that are alive (Percent Remaining).

FIG. 4 shows time (Y-axis, days [d]) to reach the study endpoint after implantation of tumor cells for mice in Group 1 (“Vehicle”, treated with saline), Group 2 (“Anti-CTLA4”, treated with anti-CTLA4 antibody), Group 3 (“INT-151”, treated with INT-151) and Group 4 (“Combination”, treated with INT-151 and anti-CTLA4). The “study endpoint” was a tumor volume of 2000 mm³ or 47 days, whichever came first.

FIG. 5 shows time (X-axis, days) to reach the study endpoint after implantation of tumor cells in mice treated with Vehicle (closed circles w/solid line), anti-CTLA-4 antibody (open squares, w/solid line), INT-151 1× weekly (open upward triangles, w/dashed line), INT-151 3× weekly (inverted open triangles w/solid line), and INT-151 and anti-CTLA-4 antibody (Combination 1× weekly, open diamonds w/dashed line; Combination 3x weekly, open circles w/dashed line). The “study endpoint” was a tumor volume of 2000 mm³ or 45 days, whichever came first. Tumor volume (mm³) is shown on the y-axis. The graph displays median values.

FIG. 6 shows tumor volume measurements over time (X-axis, days) up to day 14 in four groups of mice randomized at treatment start having median tumor volumes of 43 mm³. Treatment groups included Group 1 (“Control”, solid circles w/solid line), Group 2 (“anti-CTLA4”, open squares w/solid line), Group 3 (“INT-151”, 3 times weekly open triangles w/dashed line), and Group 4 (INT-151 with 3 times weekly anti-CTLA-4, “Combination”, inverted open triangles w/dashed line). Study endpoint was day 14 when tumor volumes in the control group exceeded 800 mm³. Tumor volume (mm³) is shown on the y-axis. The graph displays median values over time. (*p<0.05)

FIG. 7(A-I) shows immune profiles of tumors assessed using FACS analysis. The data show the changes in total macrophages (FIG. 7A), M1 and M2 macrophages (FIGS. 7B and 7C, respectively), CD4+ T cells (FIG. 7D), CD8+T cells (FIG. 7E), regulatory T cells and their ratio (FIGS. 7F and 7I, respectively), monocytic MDSC (FIG. 7H) and granulocytic MDSC (FIG. 7G) for each of the treatment groups including vehicle (“Control”, closed circles), anti-CTLA-4 (“αCTLA4”, closed squares), “INT-151 3× weekly” (closed triangles) and the “Combination” (anti-CTLA-4 plus INT-151, inverted closed triangles) in tumors (N=6). (*p<0.05; **p<0.005)

FIG. 8 shows immune profiles of peripheral blood that were assessed using FACS analysis. The data show the changes in total macrophages (FIG. 8A), M1 and M2 macrophages (FIGS. 8B and 8C, respectively), CD4+ T cells (FIG. 8D), CD8+T cells (FIG. 8E), regulatory T cells and their ratio (FIGS. 8F and 8I, respectively), monocytic MDSC (FIG. 8H) and granulocytic MDSC (FIG. 8G) for each of the treatment groups including vehicle (“Control”, closed circles), anti-CTLA-4 (“αCTLA4”, closed squares), “INT-151 3× weekly” (closed triangles) and the “Combination” (anti-CTLA-4 plus INT-151, inverted closed triangles) in peripheral blood (N=6). (*p<0.05; **p<0.005, ***p<0.0005)

### DETAILED DESCRIPTION

Presented herein are immune-stimulating peptides and checkpoint inhibitors for use as a combination therapy for treating cancer. Also presented herein are compositions comprising an immune-stimulating peptide and a checkpoint inhibitor, and uses thereof for treating cancer.

Immune-Stimulating Peptides

In some embodiments, an immune-stimulating peptide is a small peptide comprising an N-terminal or C-terminal Tyr-Gly (YG) dipeptide, or Tyr-Gly-Gly (YGG) tripeptide, repeats thereof and variants thereof. In certain embodiments, an immune-stimulating peptide comprises or consists of 1 to 200, 1 to 100, 1 to 50, 1 to 25, 1 to 10, or 1 to 5 repeats of the dipeptide YG. In some embodiments an immune-stimulating peptide comprises or consists of the amino acid sequence selected from (YG)ₙ wherein n=1, 2, 3, 4, 5, 6, 7, 8, 9, 10, 11, 12, 13, 14, 15, 16, 17, 18, 19 or 20. In certain embodiments, an immune-stimulating peptide comprises or consists of 1 to 200, 1 to 100, 1 to 50, 1 to 25, 1 to 10, or 1 to 5 repeats of the tripeptide YGG. In some embodiments an immune-stimulating peptide comprises or consists of the amino acid sequence selected from (YGG)ₙ wherein n=1, 2, 3, 4, 5, 6, 7, 8, 9, 10, 11, 12, 13, 14, 15, 16, 17, 18, 19 or 20.

An immune-stimulating peptide, in some embodiments, comprises a peptide or polypeptide of the formula (Tyr-Gly-Z)ₙ, (Tyr-Gly-Gly-Z)ₙ, (Tyr-Gly-Phe-Z)ₙ, or (Tyr-Gly-Gly-Phe-Z)ₙ, where Z is null, an amino acid, peptide or moiety linked to the carboxyl group of glycine (Gly), and n=1 to 200. In some embodiments, an immune-stimulating peptide comprises a peptide or polypeptide of the formula (Z-Tyr-Gly)ₙ, (Z-Tyr-Gly-Gly)ₙ, (Z-Tyr-Gly-Phe)ₙ, or (Z-Tyr-Gly-Gly-Phe)ₙ, where Z is an amino acid, peptide or moiety linked to the N-terminal amine of tyrosine (Tyr) and n=1 to 200. In some embodiments where Z is an amino acid, or peptide, Z is linked to the Tyr-Gly dipeptide by an amide or peptide bond. In some embodiments, Z comprises one or more aromatic amino acids. In some embodiments, Z comprises one or more Phenylalanine residues. In some embodiments, Z is Methionine (Met). In some embodiments, Z comprises the sequence Phe-Met. In certain embodiments Z comprises a peptide that is 2 to 100, 2 to 20, 2 to 15, 2 to 10, or 2 to 5 amino acids in length. In some embodiments Z comprises a peptide that is 2, 3, 4, 5, 6, 7, 8, 9, 10, 11 or 12 amino acids in length.

In certain embodiments, an immune-stimulating peptide comprises Met-enkephalin. An immune-stimulating peptide may comprise a Met-enkephalin on its C-terminus or N-terminus. In certain embodiments, Z is Met-enkephalin.

In certain embodiments, an immune-stimulating peptide comprises one or more peptides having the amino acid sequence YG and/or YGG and at least 1, at least 2, at least 10 or at least 100 aromatic amino acid residues (e.g., phenylalanine (F)) for every YG and YGG peptide present in the immune-stimulating peptide. In certain embodiments, an immune-stimulating peptide comprises one or more peptides having the amino acid sequence YG or YGG, and 1 to 1000 phenylalanine (F) residues for every YG and YGG peptide present in the immune-stimulating peptide.

In some embodiments, an immune-stimulating peptide comprises a mixture of dipeptides of the sequence YG, tripeptides of the sequence YGG and one or more phenylalanines. In some embodiments, an immune-stimulating peptide comprises or consists of a mixture of dipeptides (YG)ₐ, tripeptides (YGG)_{b} and (phenylalanine)_{c} present at a ratio of a:b:c where a is 100 to 300, b is 1 to 10 and c is 500 to 200,000. In some embodiments, an immune-stimulating peptide is a peptide disclosed in Gottlieb M. et al. (1991) Annals of Internal Medicine 115(2):84-91.

In certain embodiments, an immune-stimulating peptide is 2 to 100,000, 2 to 10,000, 2 to 1000, 2 to 100, 2 to 20, 2 to 15, 2 to 10, or 2 to 5 amino acids in length. In some embodiments, an immune-stimulating peptide is 2, 3, 4, 5, 6, 7, 8, 9, 10, 11, 12, 13, 14 or 15 amino acids in length.

A “peptide” refers to two, or more, amino acids linked by an amide or equivalent bond. Peptides can sometimes form intra or intermolecular disulfide bonds. Peptides can also form higher order structures, such as multimers or oligomers, with the same or different peptides, or other molecules.

In certain embodiments, an immune-stimulating peptide comprises one or more modified amino acid, amino acid analogues or amino acid variants. In certain embodiments, a modified amino acid includes one or more amino acids having a side chain, N-terminus, or C-terminus that is chemically altered or derivatized. Non-limiting examples of modified amino acids include amino acids modified by acetylation, acylation, phosphorylation, glycosylation, myristoylation, amidation, hydroxylation (e.g., aspartic acid/asparagine hydroxylation), phosphopantethane attachment, methylation, methylthiolation, prenylation, addition of an intein, ADP-ribosylation, bromination, citrullination, deamination, dihdroxylation, formylation, geranyl-geranylation, glycation or palmitoylation. Non-limiting examples of amino acid analogues include D-amino acids, β-amino acids, α-amino-n-butyric acid, norvaline, dehydroalanine, norleucine, alloisoleucine, t-leucine, α-amino-n-heptanoic acid, pipecolic acid, α,β-diaminopropionic acid, α,γ-diaminobutyric acid, ornithine, allothreonine, homocysteine, homoserine, β-alanine, β-amino-n-butyric acid, β-aminoisobutyric acid, γ-aminobutyric acid, α-aminoisobutyric acid, isovaline, sarcosine, N-ethyl glycine, N-propyl glycine, N-isopropyl glycine, N-methyl alanine, N-ethyl alanine, N-methyl β-alanine, N-ethyl β-alanine, isoserine, and α-hydroxy-γ-aminobutyric acid. An immune-stimulating peptide may include one or more D-amino acids substituted for L-amino acids. In certain embodiments, an immune-stimulating peptide comprises an amino acid analogue or unnatural amino acid described in Yamaguchi et al. (2003) Bioscience, Biotechnology, and Biochemistry 67(10): 2269-2272. In certain embodiments, an immune-stimulating peptide comprises an amino acid analogue or unnatural amino acid described in Haug B, et al. (2016) J. Med. Chem. 59:2918-2927. In some embodiments, an immune-stimulating peptide comprises one or more bulk amino acids, or bulk aromatic amino acids, non-limiting examples of which include Dip (3,3-diphenylalanine), Bip (biphenylalanine), Ath (9-anthracenylalanine), Nal (1-naphthylalanine), Dap (2,3-diaminopropionic acid) and Dab (2,4-diaminobutanoic acid). In certain embodiments, one or more modified amino acid, amino acid analogues, amino acid variants, D-amino acids, bulk amino acids or synthetic amino acids are introduced into an immune-stimulating peptide to increase the in vivo stability or half-life of an immune-stimulating peptide.

In some embodiments, an immune-stimulating peptide comprises an amino acid sequence selected from YG, YGG, YGGF, YGGDip, YGGBip, YGGAth, YGGDap, YGGDab, YGGNal, and YGGW, YGGFGGFG, YGGFGGDipG, YGGFGGBipG, YGGFGGAthG, YGGFGGDapG, YGGFGGDabG, YGGFGGNalG, YGGWGGFG, YGGWGGWG, YGGFM, YGGDipM, YGGBipM, YGGAthM, YGGDapM, YGGDabM, YGGNalM, YGGWMYGAF, YGAW, YGAibF, YGAibW, YGAibFM, YGADipM, YGABipM, YGAAthM, YGADapM, YGADabM, YGANalM, repeats thereof, and combinations thereof. In some embodiments, an immune-stimulating peptide comprises or consists of an amino acid sequence selected from YGGF, YGGDip, YGGBip, YGGAth, YGGDap, YGGDab, YGGNal, and YGGW. In certain embodiments, an immune-stimulating peptide comprises or consists of an amino acid sequence selected from YGGFGGFG, YGGFGGDipG, YGGFGGBipG, YGGFGGAthG, YGGFGGDapG, YGGFGGDabG, YGGFGGNalG, YGGWGGFG and YGGWGGWG. In certain embodiments, an immune-stimulating peptide comprises or consists of an amino acid sequence selected from YGGFM, YGGDipM, YGGBipM, YGGAthM, YGGDapM, YGGDabM, YGGNalM and YGGWM. In certain embodiments, an immune-stimulating peptide comprises or consists of an amino acid sequence selected from YGAF, YGAW, YGAibF, YGAibW, YGAibFM, YGADipM, YGABipM, YGAAthM, YGADapM, YGADabM and YGANalM.

In some embodiments, an immune-stimulating peptide comprises or consists of an amino acid sequence selected from YAGF, YAGW, YAibGF, YAibGFM, ACYGGGACG, ACYGGFACG, ACYGGBipACG, ACYGGDipACG, ACYGGAthACG, ACYGGDabACG, ACYGGDapACG and ACYGGNalACG. In some embodiments, an immune-stimulating peptide includes cyclic structures such as an end-to-end amide bond between the amino and carboxy-terminus of the peptide or intra- or inter-molecular disulfide bond. In some embodiments, an immune-stimulating peptide may be cyclic by adding appropriate groups or linker sequences to generate a cyclic peptide (e.g. ACYGGGWCG containing reactive cysteines for dimerization or a bridging sequence), or a sequence that generates a stapled peptide. In some embodiments, an immune-stimulating peptide comprises or consists of a cyclic peptide, non-limiting examples of which include cyclic peptide comprising the amino acid sequence ACYGGGACG, ACYGGFACG, ACYGGBipACG, ACYGGDipACG, ACYGGAthACG, ACYGGDabACG, ACYGGDapACG and ACYGGNalACG where the two cysteine residues of each peptide are linked by a disulfide bridge.

Further, in certain embodiments, an immune-stimulating peptide comprises a suitable moiety, often for the purpose of increasing in vivo half-life, non-limiting examples of which include polyethylene glycol (PEG), PEG mimetic substitutes, dextran, a polypeptide (e.g., albumin, an immunoglobulin Fc region, a portion thereof, or the like), a sugar, a carbohydrate (e.g., added by glycosylation), a lipid (e.g., added by myristoylation), a nucleic acid, a carrier or vehicle described in U.S. Pat. No. 6,660,843 (which is incorporated herein by reference), the like and/or combinations thereof. For example, where an immune-stimulating peptide comprises the formula N-Tyr-Gly-Z, where N is the N-terminal amine of Tyrosine, Z may comprise or consist of a moiety selected from PEG, PEG mimetic substitutes, dextran, a polypeptide (e.g., albumin, an immunoglobulin Fc region, a portion thereof, or the like), a sugar, a carbohydrate (e.g., added by glycosylation), a lipid (e.g., added by myristoylation), a nucleic acid, a carrier and vehicle described in U.S. Pat. No. 6,660,843. In some embodiments where an immune-stimulating peptide comprises the formula Z-Tyr-Gly-COOH, where COOH is the carboxyl group of glycine, Z may comprise or consist of a moiety selected from PEG, PEG mimetic substitutes, dextran, a polypeptide (e.g., albumin, an immunoglobulin Fc region, a portion thereof, or the like), a sugar, a carbohydrate (e.g., added by glycosylation), a lipid (e.g., added by myristoylation), a nucleic acid, a carrier and vehicle described in U.S. Pat. No. 6,660,843. In some embodiments, an immune-stimulating peptide comprises one, two or more bulky amino acids that are covalently linked to a Tyr-Gly dipeptide portion of an immune-stimulating peptide to provide enhanced stability. Non-limiting examples of bulky amino acids include natural and synthetic amino acids, or derivatives thereof, that comprise an aromatic moiety. In certain embodiments, an immune-stimulating peptide comprises a suitable immunogen, non-limiting examples of which include Keyhole Limpet Hemocyanin (KLH), Concholepas Hemocyanin (CCH), Blue Carrier Immunogenic Protein, Bovine Serum Albumin (BSA), human serum albumin (HSA), ovalbumin (OVA), the like, modifications thereof or combinations thereof. Therefore, Z may comprise one or more bulky amino acids (e.g., 1, 2, 3, 4, or at least 5 bulky amino acids) and/or a suitable immunogen. Accordingly, in some embodiments, a suitable moiety or immunogen is linked to, or attached to the N-terminal amine of a Tyr-Gly dipeptide portion of an immune-stimulating peptide. In some embodiments, a suitable moiety or immunogen is linked to, or attached to the C-terminal carboxyl group of a Tyr-Gly dipeptide portion of an immune-stimulating peptide.

In certain embodiments, a Tyr-Gly dipeptide portion of an immune-stimulating peptide is not modified or altered. In certain embodiments, a Tyr-Gly dipeptide portion of an immune-stimulating peptide comprises a free, unaltered and unmodified N-terminus, and/or a free, unaltered and unmodified C-terminus (i.e., carboxy terminus, i.e., —COOH). In certain embodiments, the R-groups of a Tyr-Gly dipeptide of an immune-stimulating peptide are not altered or modified.

In some embodiments, an immune-stimulating peptide comprises a microsphere or nanosphere. For example, one or more immune-stimulating peptides may be attached, covalently or non-covalently to a microsphere or nanosphere, or alternatively encased or contained within a spherical shell of a microsphere or nanosphere. In some embodiments, one or more immune-stimulating peptides are attached to the surface of a microsphere or nanosphere. A microsphere can be made of any suitable material. A microsphere or nanosphere, in some embodiments, comprises a biodegradable or resorbable polymer that is suitable for administration to humans. In certain embodiments, a microsphere or nanosphere is relatively inert.

Checkpoint Inhibitors

In certain embodiments, a checkpoint inhibitor is an antibody or compound that inhibits, blocks, ameliorates, or suppresses signal transduction through Cytotoxic T-lymphocyte protein 4 (CTLA-4) (e.g., see UniProtKB: P16410) or Programmed Cell Death 1 (PD1, or PD-1), also known as PCD1 and CD279 (e.g., see UniProtKB: Q15116). In certain embodiments, a checkpoint inhibitor is an antibody or compound that inhibits, blocks, ameliorates, or suppresses activation of a T-cell mediated by signal transduction through CTLA-4 or PD1. In certain embodiments, a checkpoint inhibitor is an antibody or compound that inhibits, blocks, ameliorates, or suppresses binding of CTLA-4 to one of its cognate ligands, non-limiting examples of which include CD86 (B7-2), CD80 (B7-1) and B7-Related protein (ICOS Ligand, CD275). In certain embodiments, a checkpoint inhibitor is an antibody or compound that inhibits, blocks, ameliorates, or suppresses binding of PD-1 to one of its cognate ligands, non-limiting examples of which include PD-L1 and PD-L2.

In some embodiments, a checkpoint inhibitor is an antibody that binds specifically to PD-1, non-limiting examples of which include Nivolumab (BMS-936558/MDX-1106/ONO-4538)(Brahmer J R, et al. (2010) J Clin Oncol. 28(19):3167-75); Lambrolizumab (alias: MK3475 (Merck), Pembrolizumab, Keytruda)(U.S. Pat. No. 8,952,136); Pidilizumab (CT-011, CureTech)(Westin J R, et al., (2014) Lancet Oncol. 15(1):69-77); AMP-514 (Medlmmune/AZ, alias:MEDI0680)(WO/2012/145493); AMP-224 (GSK, alias: B7-DCIg, a PDL2-Fc fusion soluble receptor described in WO2010/027827 and WO2011/066342) and Pembrolizumab (Keytruda). In some embodiments, a checkpoint inhibitor is AUNP 12 (Aurigene and Pierre Fabre Pharmaceuticals; US Patent Publication No. US20110318373). In some embodiments, a checkpoint inhibitor is an antibody that to PD-L1, non-limiting examples of which include BMS-936559 (Bristol-Myers Squibb)(WO/2007/005874); MPDL3280A (Genentech)(RG7446; WO/2010/077634); MedI-4736 (Medimmune, previously Pfizer), MSB0010718C (EMD Serono)(WO/2013/79174); Atezolizumab (Tecentriq), Avelumab (Bavencio), Durvalumab (Imfinzi) and MDX-1105 (WO/2007/005874).

In some embodiments, a checkpoint inhibitor is an antibody that binds specifically to CTLA-4, non-limiting examples of which include Ipilimumab (Bristol-Myers Squibb, Yervoy®)(Int. Appl. No. PCT/US1999/28739 (published as WO/2000/32231), U.S. Pat. Nos. 5,811,097, 5,855,887, 6,051,227, and 6,207,156)); and Tremelimumab (formerly CP-675,206)(Medimmune, previously Pfizer)(Calabrò L, et al., (2013) Lancet Oncol. 14(11):1104-11; and Camacho, L H et al, (2004) J. Clin. Oncol., Suppl. (S), vol. 22, no. 14, pages 164S-164S).

Additional non-limiting examples of checkpoint inhibitors include Urelumab (BMS), Anti-OX40 AB-(GSK/MDACC), LAG3—BMS986016 (BMS), T-cell immunoglobulin and mucin domain containing 3 (TIM3) (T-cell activation), Glucocorticoid-induced tumor necrosis factor receptor family related protein (GITR) (T-reg)-TRX 518 GITR, MK4166 (Merck), CD27 (T cell memory, APC activation)—CDX1127 CellDex, KIR (activate T and NK cells)-Lirilumab (BMS), IPH2101 Innate Pharma, TIGIT (Rigel Pharma), ICOS or CD278 (inducible T-cell co-stimulator) Jounce Therapeutics, IDO, T-cells (Epacadostat, Incyte; Indoximod, NewLink genetics; BMS 986205; NLG802, NewLink Genetics), CD137 T-cell proliferation (Pieris Pharmaceuticals), and VISTA T-cell proliferation primarily hematopoietic cells.

Distinguishable Identifiers, Detection & Diagnosis

In some embodiments an immune-stimulating peptide comprises one or more distinguishable identifiers. Any suitable distinguishable identifier and/or detectable identifier can be used for an immune-stimulating peptide, composition or method described herein. In certain embodiments a distinguishable identifier can be directly or indirectly associated with (e.g., bound to) an immune-stimulating peptide. For example, a distinguishable identifier can be covalently or non-covalently bound to an immune-stimulating peptide. In some embodiments a distinguishable identifier is reversibly associated with an immune-stimulating peptide. In certain embodiments a distinguishable identifier that is reversibly associated with an immune-stimulating peptide can be removed from an immune-stimulating peptide using a suitable method (e.g., by increasing salt concentration, denaturing, washing, adding a suitable solvent and/or by heating).

In some embodiments a distinguishable identifier is a detectable label. In some embodiments an immune-stimulating peptide comprises a detectable label, non-limiting examples of which include a radiolabel (e.g., an isotope), a metallic label, a fluorescent label, a chromophore, a chemiluminescent label, an electrochemiluminescent label (e.g., Origen™), a phosphorescent label, a quencher (e.g., a fluorophore quencher), a fluorescence resonance energy transfer (FRET) pair (e.g., donor and acceptor), a dye, a protein (e.g., an enzyme; e.g., alkaline phosphatase and horseradish peroxidase), an antibody, an antigen or part thereof, a linker, a member of a binding pair), an enzyme substrate, a small molecule (e.g., biotin, avidin), a mass tag, quantum dots, nanoparticles, the like or combinations thereof. Any suitable fluorophore can be used as a detectable label. A detectable label can be detected and/or quantitated by a variety of suitable techniques thereby allowing tracking, detection and quantitation of an immune-stimulating peptide. Accordingly, in some embodiments, a composition or kit disclosed herein comprises an immune-stimulating peptide comprising a distinguishable identifier or a detectable label. Further, presented herein is a method of diagnosing or detecting a cancer in a subject, or determining an amount of a cancer in a subject, where the method comprises administering an immune-stimulating peptide to the subject, optionally in combination with a checkpoint inhibitor, wherein the immune-stimulating peptide comprises a distinguishable identifier or a label, and detecting or quantifying an amount of the immune-stimulating peptide that is bound or associated with the cancer.

Subjects

The term “subject” refers to animals, typically mammalian animals. In some embodiments a subject is a mammal. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a subject is a primate. In some embodiments a subject is a non-human primate. In some embodiments a subject is a human. A mammal can be any age or at any stage of development (e.g., an adult, teen, child, infant, or a mammal in utero). A mammal can be male or female. In certain embodiments a mammal can be an animal disease model, for example, animal models used for the study of cancer.

In certain embodiments a subject has or is suspected of having a cancer. In certain embodiments a subject is at risk of developing a cancer. Subjects at risk of developing a cancer can be subjects in high-risk groups who can be identified by a medical professional. Non-limiting examples of subjects at risk of cancer include chronic smokers, overweight individuals, human subjects over the age of 60, subjects with a family history of cancer, subjects having certain gene mutations that are associated with certain cancers, subjects infected with, or previously infected with certain viruses associated with the development of certain cancers, subjects exposed to known carcinogens, subjects exposed to excessive radiation (e.g., UV radiation, alpha, beta, or gamma radiation), subjects having chronic inflammation, the like, or combinations thereof. In some embodiments a subject or mammal is “at risk” of cancer metastasis. Certain cancers are known to be metastatic or have a high probability of metastasis depending on the cancer type, stage, tissue or origin, and/or age, sex or health condition of a subject. Metastatic disease typically occurs when patients show relapse after standard of care therapy. A subject at risk can be readily identified by a medical professional (e.g., a doctor, or an oncologists).

Methods of Treatment

In some embodiments, a method described herein comprises a method of treating a cancer in a subject that has, or is suspected of having, a cancer. In certain embodiments, a method of treating a cancer comprises blocking, inhibiting, ameliorating, abrogating, or suppressing growth, metastasis or viability of a cancer (e.g., cancer cells, e.g., a tumor). In some embodiments, a method of treating a cancer comprises a method of inhibiting, blocking, ameliorating, reducing or suppressing growth or viability of a cancer in a subject. In some embodiments, a method of treating a cancer comprises inhibiting, blocking, ameliorating, reducing or suppressing metastasis of a cancer in a subject. In certain embodiments, a method of treating a cancer in a subject comprises an attempt to inhibit, block, ameliorate, reduce or suppress growth, viability or metastasis of a cancer in a subject, with a reasonable expectation of success. In certain embodiments, a method of treating a cancer comprises inducing necrosis, death or apoptosis of one or more cancer cells in a subject. In certain embodiments, a method of treating a cancer comprises inhibiting, reducing the severity of, delaying the onset of, suppressing, ameliorating, or abrogating one or more symptoms associate with a cancer. In certain embodiments, a method of treating a cancer comprises prolonging or sustaining life, or improving the quality of life of a subject having a cancer, compared to the length or quality of life that the subject would have in the absence of a cancer treatment.

In some embodiments, a method of treating a subject comprises administering to the subject a pharmaceutically effective amount of a composition disclosed herein. In some embodiments, a method of treating a subject comprises administering to a subject in need thereof a therapeutically effective amount of an immune-stimulating peptide and a therapeutically effective amount of a checkpoint inhibitor. In some instances a cancer in a subject may become resistant to therapy with a checkpoint inhibitor or chemotherapy. Alternatively, an immune-stimulating peptide may be administered to supplement and enhance the activity of checkpoint inhibitor or certain chemotherapies. Accordingly, in certain embodiments, a method of treating a subject comprises, (a) providing a subject undergoing treatment with, or previously treated with, a checkpoint inhibitor or chemotherapeutic agent, and (b) administering to the subject a therapeutically effective amount of an immune-stimulating peptide and a therapeutically effective amount of a checkpoint inhibitor or chemotherapeutic agent. In certain embodiments, a method of treating a subject comprises, (a) providing a subject having or suspected of having a cancer, wherein the subject is resistant to, or unresponsive to, treatment with a checkpoint inhibitor, and (b) administering to the subject a therapeutically effective amount of an immune-stimulating peptide and a therapeutically effective amount of a checkpoint inhibitor. The checkpoint inhibitor administered in (b) above, may be the same or a different checkpoint inhibitor as the checkpoint inhibitor previously administered to the subject.

In some embodiments, an immune-stimulating peptide and a checkpoint inhibitor are administered to a subject at substantially the same time. In some embodiments, an immune-stimulating peptide and a chemotherapeutic agent are administered to a subject at substantially the same time. In certain embodiments, an immune-stimulating peptide and a checkpoint inhibitor or chemotherapeutic agent are administered to a subject at different times. In certain embodiments, an immune-stimulating peptide and a checkpoint inhibitor are administered within 5 minutes, within 10 minutes, within 15 minutes, within 30 minutes, within 45 minutes, within 60 minutes, or within 120 of each other. In certain embodiments, an immune-stimulating peptide and the checkpoint inhibitor are administered within 1 hour, within 2 hours, within 3 hours, within 4 hours, within 5 hours, within 6 hours, within 8 hours, within 10 hours, within 12 hours or within 24 hours of each other. In certain embodiments, an immune-stimulating peptide and the checkpoint inhibitor are administered within 1 to 3 days of each other, or within 1, 2 or 3 days of each other.

In certain embodiments, a method of treating a cancer in a subject comprises administering a therapeutically effective amount of an immune-stimulating peptide and a therapeutically effective amount of a checkpoint inhibitor to a subject in need thereof, in combination with another anti-cancer therapy, non-limiting examples of which include a T-cell activating agent, an adjuvant, an anti-cancer vaccine, a radiation treatment, or a chemotherapeutic agent or therapy. In some embodiments, a T-cell activating agent is an antibody that binds to CD3, OX40, GITR, CD137 (41BB), CD27, HVEM, LAG-3, TIM3, VISTA or BTLA.

Any suitable chemotherapeutic agent can be used for a method described herein. In some embodiments a chemotherapeutic agent comprises or consists of an alkylating agent, an anthracycline, cytoskeletal disruptors, epothilones (e.g., epothilone), histone deacetylase inhibitors (e.g., vorinostat, romidepsin), inhibitors of topoisomerase I (e.g., irinotecan, topotecan), inhibitors of topoisomerase II (e.g., etoposide, teniposide, tafluposidean), kinase inhibitors, peptide antibiotics (e.g., bleomycin, actinomycin), platinum-based agents (e.g., carboplatin, cisplatin, oxaliplatin), compounds targeting DNA repair enzyme poly-ADP ribose polymerase-1, Parp inhibitors, retinoids (e.g., tretinoin, alitretinoin, bexarotene), vinca alkaloids and derivatives (e.g., vinblastine, vincristine, vindesine, vinorelbine), anti-metabolites, plant extracts, plant alkaloids, nitrosourea, hormone, nucleoside or nucleotide analog and combinations thereof.

In some embodiments, a chemotherapeutic agent comprises an alkylating anti-neoplastic agent (e.g., an alkylating anti-neoplastic agent). An alkylating antineoplastic agent is a class of chemotherapeutic agents that work, in part, by attaching an alkyl group (e.g., CnH2n+1) to DNA, a process known as alkylation. Some alkylating antineoplastic agents are administered as a pro-drug that is converted in vivo to an active alkylating agent. An alkylating antineoplastic agent often alkylates a guanine base of DNA. Alkylating antineoplastic agents are most effective on proliferating cells (e.g., cancer cells) which, in general, proliferate faster and with less error-correcting than healthy cells. Non-limiting examples of alkylating anti-neoplastic agents include Altretamine (hexamethylmelamine, HEXALEN®), Busulfan, Carmustine (BCNU), Chlorambucil, Cyclophosphamide, Dacarbazine (DTIC), Fotemustine, Ifosfamide, Lomustine (CCNU), Mechlorethamine, Melphalan, Procarbazine, semustine (MeCCNU), Streptozotocin, Temozolomide, Thiotepa (triethylenethio-phosphoramide), Carboplatin, Cisplatin, Oxaliplatin, monofunctional alkylators, nitrosoureas, temozolomide, the like or combinations thereof.

In some embodiments, a chemotherapeutic agent comprises a DNA intercalating agent which is often an agent that attaches or binds to DNA or RNA. Non-limiting examples of a DNA intercalating agent include acrolein, anthracycline, phosphoramide, Actinomycin D, bleomycin, idarubicin, daunorubicin, doxorubicin, elsamicin A, epirubicin, ethidium, m-AMSA, mitoxantrone, doxorubicin (Adriamycin, Doxil, Myocet, hydroxydaunorubicin, hydroxydaunomycin), Epirubicin, Idarubicin, Valrubicin, TAS-103, MLN944 (XR5944), Obatoclax, mechlorethamine, methotrexate, 6-mercaptopurine, thioguanine, 5-fluorouracil, cytosine arabinoside, 5-azacytidine (5-AZC) and 5-azacytidine related compounds, mithramycin, mitomycin C, hydroxyurea, carboplatin, oxiplatin, mitotane, a taxane, vinblastine, vincristine, dibromomannitol, gemcitabine, pemetrexed, the like or a combination thereof.

In some embodiments, a chemotherapeutic agent comprises a cytoskeletal disruptor. Non-limiting examples of cytoskeletal disruptors (e.g., taxanes) include paclitaxel, taxol, and docetaxel.

In some embodiments, a chemotherapeutic agent comprises a kinase inhibitor. Non-limiting examples of kinase inhibitors include bortezomib, erlotinib, gefitinib, imatinib, vemurafenib, vismodegib, the like, analogs and derivatives thereof.

In some embodiments, a chemotherapeutic agent comprises one or more nucleotide analogs. Non-limiting examples of nucleotide analogs include azacitidine, azathioprine, capecitabine, cytarabine, doxifluridine, fluorouracil, gemcitabine, hydroxyurea, mercaptopurine, methotrexate, tioguanine (formerly thioguanine), the like, analogs and derivatives thereof.

In some embodiments, a chemotherapeutic agent comprises one or more molecules that target DNA repair enzyme poly-ADP ribose polymerase-1 (PARP) and inhibit DNA repair. Non-limiting examples of PARP inhibitors are olaparib, rucaparib, niraparib, veliparib, talazoparib and the like, analogs and derivatives thereof.

Cancers & Metastasis

In some embodiments, a method or composition disclosed herein is for use in treating a subject having or suspected of having a cancer. In some embodiments, a cancer is a neoplasm or tumor. A cancer may be metastatic or non-metastatic. In certain embodiments, a cancer is a malignant and/or a metastatic cancer. Non-limiting examples of a cancer include melanoma, lymphoma (e.g., Hodgkin lymphoma, non-Hodgkin lymphoma, a B-cell neoplasm, a T-cell neoplasm), leukemia, reticuloendothelial hyperplasia (e.g., reticuloendothelial neoplasia), lymphatic neoplasia, hematopoietic neoplasia, myeloma, multiple myeloma, an immunodeficiency-associated lymphoproliferative disorder, adenoma, adenocarcinoma, sarcoma (non-limiting examples of which include a lymphosarcoma, liposarcoma, osteosarcoma, chondrosarcoma, leiomyosarcoma, rhabdomyosarcoma, fibrosarcoma, the like or combinations thereof), carcinoma, breast cancer, colorectal cancer, gastrointestinal cancer, hepatocellular cancer, lung cancer, bone cancer, renal cancer, bladder cancer, hepatoma, neuroblastoma, retinoblastoma, astrocytoma, glioma, glioblastoma, medulloblastoma, meningioma, oligodendrocytoma, cervical cancer, testicular cancer, ovarian cancer, mesothelioma, esophageal cancer, pancreatic cancer, prostate cancer, the like or combinations thereof.

In some embodiments, a cancer comprises a metastatic melanoma. In some embodiments a cancer comprises a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin, lung, biliary tract, or hematologic neoplasia, tumor, or cancer. In some embodiments a cancer comprises a solid cellular mass. In certain embodiments a malignant cancer comprises or consists of a fibrosarcoma, myxosarcoma, liposarcoma, chondrosarcoma, osteosarcoma, chordoma, malignant fibrous histiocytoma, hemangiosarcoma, angiosarcoma, lymphangiosarcoma, mesothelioma, leiomyosarcoma, rhabdomyosarcoma, squamous cell carcinoma, epidermoid carcinoma, malignant skin adnexal tumor, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma, glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, oat cell carcinoma, malignant pheochromocytoma, islet cell carcinoma, malignant carcinoid, retinoblastoma, chemodectoma, paraganglioma, malignant carcinoid, malignant paraganglioma, melanoma, malignant schwannoma, merkel cell neoplasm, cystosarcoma phylloides, Wilms tumor, malignant ovarian tumors, malignant testicular tumors, the like, or combinations thereof. In certain embodiments a cancer comprises a carcinoma, sarcoma, lymphoma, leukemia, adenoma, adenocarcinoma, melanoma, glioma, glioblastoma, medulloblastoma, Kaposi sarcoma, meningioma, neuroblastoma, retinoblastoma, astrocytoma, oligodendrocytoma, reticuloendothelial, lymphatic or haematopoietic neoplasia, tumor, cancer or malignancy. In certain embodiments, a sarcoma comprises a lymphosarcoma, liposarcoma, osteosarcoma, chondrosarcoma, leiomyosarcoma, rhabdomyosarcoma or fibrosarcoma.

In some embodiments a leukemia is an acute lymphocytic leukemia (ALL), acute myeloid leukemia (AML), chronic lymphocytic leukemia (CLL), chronic myeloid leukemia (CML), or chronic myelomonocytic leukemia (CMML).

Methods of Cell Expansion

In some embodiments, presented herein are methods of expanding T-cells ex vivo or in vitro. In certain embodiments, T-cells that are expanded by a method described herein can be used for adoptive cell therapy for the treatment of cancer. T-cells that are expanded ex vivo or in vitro can be native T-cells and/or genetically modified T-cells. In some embodiments, T-cells are obtained or isolated from a subject (e.g., a human subject). In some embodiments, T-cells are obtained or isolated from a subject who has, or is suspected of having, a cancer. In some embodiments, T-cells are tumor infiltrating lymphocytes (TILs) extracted from a patient's tumor. In some embodiments, T-cells are isolated from peripheral blood mononuclear cell (PBMC) populations using a suitable method. In some embodiments, T-cells that are expanded in vitro, or ex vivo, are genetically modified to express a T-cell receptor (e.g., CAR-T cells).

In some embodiments, a method of expanding T-cells comprises a method of inducing mitotic division, growth and/or proliferation of the T-cells. In certain embodiments, a method of expanding T-cells comprises contacting a population of T-cells in vitro or ex vivo, with an effective amount of one or more immune stimulating peptides described herein. In certain embodiments, a method of expanding T-cells comprises contacting a population of T-cells in vitro or ex vivo, with an effective amount of one or more immune stimulating peptides described herein and with an effective amount of one or more checkpoint inhibitors. In certain embodiments, a method of expanding T-cells comprises contacting a population of T-cells in vitro or ex vivo, with an effective amount of one or more immune stimulating peptides described herein and with an antigen presented by a dendritic cell or a carrier.

In some embodiments, a method of expanding T-cells comprises contacting a population of T-cells with an effective amount of one or more immune stimulating peptides, allowing the T-cells to expand for 4 to 30 days (4 to 14 days, or 7 to 14 days) and re-introducing some or all of the expanded T-cells into a subject. In some embodiments, a method of expanding T-cells comprises (i) obtaining T-cells from a donor subject who has, or is suspected of having cancer, (ii) contacting the T-cells, in vitro or ex vivo, with an effective amount of one or more immune stimulating peptides, (iii) allowing the T-cells to expand for 4 to 30 days (4 to 14 days, or 7 to 14 days) and (iv) re-introducing some or all of the expanded T-cells back into the donor subject. In certain embodiments, one or more steps of a method of expanding T-cells as described herein are performed under aseptic conditions.

Compositions, Administration and Dosing

In some embodiments, a composition comprises one or more immune-stimulating peptides. In some embodiments, a composition comprises one or more checkpoint inhibitors. In some embodiments, a composition comprises one or more immune-stimulating peptides and/or one or more checkpoint inhibitors. In certain embodiments, a composition described herein comprises one or more immune-stimulating peptides, one or more checkpoint inhibitors and an adjuvant. In certain embodiments, a composition described herein comprises one or more immune-stimulating peptides, one or more checkpoint inhibitors and a cancer vaccine. In certain embodiments, a pharmaceutical composition comprises one or more immune-stimulating peptides, one or more checkpoint inhibitors and a chemotherapeutic agent. In certain embodiments, a composition described herein is a pharmaceutical composition suitable for administration to a human. In some embodiments, a pharmaceutical composition comprises one or more suitable pharmaceutical carriers. In some embodiments, a pharmaceutical composition is sterile. In certain embodiments, a composition described herein comprises one or more immune-stimulating peptides, one or more checkpoint inhibitors and an adjuvant. In certain embodiments, a composition described herein comprises one or more immune-stimulating peptides, one or more checkpoint inhibitors and a cancer vaccine. In certain embodiments, a pharmaceutical composition comprises one or more immune-stimulating peptides, one or more checkpoint inhibitors and a chemotherapeutic agent.

A pharmaceutical composition can be formulated for a suitable route of administration. In some embodiments a pharmaceutical composition is formulated for subcutaneous (s.c.), intradermal, intramuscular, intraperitoneal and/or intravenous (i.v.) administration. In certain embodiments, a pharmaceutical composition can contain formulation materials for modifying, maintaining, or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates (e.g., phosphate buffered saline) or suitable organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); solvents (such as glycerin, propylene glycol or polyethylene glycol); diluents; excipients and/or pharmaceutical adjuvants (e.g., as described in Remington '95 or Remington 2013).

In certain embodiments, a pharmaceutical composition comprises a suitable excipient, non-limiting examples of which include anti-adherents (e.g., magnesium stearate), a binder, fillers, monosaccharides, disaccharides, other carbohydrates (e.g., glucose, mannose or dextrin), sugar alcohols (e.g., mannitol or sorbitol), coatings (e.g., cellulose, hydroxypropyl methylcellulose (HPMC), microcrystalline cellulose, synthetic polymers, shellac, gelatin, corn protein zein, enterics or other polysaccharides), starch (e.g., potato, maize or wheat starch), silica, colors, disintegrants, flavors, lubricants, preservatives, sorbents, sweeteners, vehicles, suspending agents, surfactants and/or wetting agents (such as pluronics, PEG, sorbitan esters, polysorbates such as polysorbate 20, polysorbate 80, triton, tromethamine, lecithin, cholesterol, tyloxapal), stability enhancing agents (such as sucrose or sorbitol), and tonicity enhancing agents (such as alkali metal halides, sodium or potassium chloride, mannitol, sorbitol), and/or any excipient disclosed in Remington '95 or Remington 2013. The term “binder” as used herein refers to a compound or ingredient that helps keeps a pharmaceutical mixture combined. Suitable binders for making pharmaceutical formulations are often used in the preparation of pharmaceutical tablets, capsules and granules and are known to those skilled in the art.

In some embodiments a pharmaceutical composition comprises a suitable pharmaceutically acceptable additive and/or carrier. Non-limiting examples of suitable additives include a suitable pH adjuster, a soothing agent, a buffer, a sulfur-containing reducing agent, an antioxidant and the like. Non-limiting examples of a sulfur-containing reducing agent include those having a sulfhydryl group (e.g., a thiol) such as N-acetylcysteine, N-acetylhomocysteine, thioctic acid, thiodiglycol, thioethanolamine, thioglycerol, thiosorbitol, thioglycolic acid and a salt thereof, sodium thiosulfate, glutathione, and a C1-C7 thioalkanoic acid. Non-limiting examples of an antioxidant include erythorbic acid, dibutylhydroxytoluene, butylhydroxyanisole, alpha-tocopherol, tocopherol acetate, L-ascorbic acid and a salt thereof, L-ascorbyl palmitate, L-ascorbyl stearate, sodium bisulfite, sodium sulfite, triamyl gallate and propyl gallate, as well as chelating agents such as disodium ethylenediaminetetraacetate (EDTA), sodium pyrophosphate and sodium metaphosphate. Furthermore, diluents, additives and excipients may comprise other commonly used ingredients, for example, inorganic salts such as sodium chloride, potassium chloride, calcium chloride, sodium phosphate, potassium phosphate and sodium bicarbonate, as well as organic salts such as sodium citrate, potassium citrate and sodium acetate.

The pharmaceutical compositions used herein can be stable over an extended period of time, for example on the order of months or years. In some embodiments a pharmaceutical composition comprises one or more suitable preservatives. Non-limiting examples of preservatives include benzalkonium chloride, benzoic acid, salicylic acid, thimerosal, phenethyl alcohol, methylparaben, propylparaben, chlorhexidine, sorbic acid, hydrogen peroxide, the like and/or combinations thereof. A preservative can comprise a quaternary ammonium compound, such as benzalkonium chloride, benzoxonium chloride, benzethonium chloride, cetrimide, sepazonium chloride, cetylpyridinium chloride, or domiphen bromide (BRADOSOL®). A preservative can comprise an alkyl-mercury salt of thiosalicylic acid, such as thimerosal, phenylmercuric nitrate, phenylmercuric acetate or phenylmercuric borate. A preservative can comprise a paraben, such as methylparaben or propylparaben. A preservative can comprise an alcohol, such as chlorobutanol, benzyl alcohol or phenyl ethyl alcohol. A preservative can comprise a biguanide derivative, such as chlorohexidine or polyhexamethylene biguanide. A preservative can comprise sodium perborate, imidazolidinyl urea, and/or sorbic acid. A preservative can comprise stabilized oxychloro complexes, such as known and commercially available under the trade name PURITE®. A preservative can comprise polyglycol-polyamine condensation resins, such as known and commercially available under the trade name POLYQUART® from Henkel KGaA. A preservative can comprise stabilized hydrogen peroxide. A preservative can be benzalkonium chloride. In some embodiments a pharmaceutical composition is free of preservatives.

In some embodiments a composition or pharmaceutical composition is substantially free of contaminants (e.g., blood cells, platelets, polypeptides, minerals, blood-borne compounds or chemicals, virus, bacteria, other pathogens, toxin, and the like). In some embodiments a composition, pharmaceutical composition is substantially free of serum and serum contaminants (e.g., serum proteins, serum lipids, serum carbohydrates, serum antigens and the like). In some embodiments a composition or pharmaceutical composition is substantially free of a pathogen (e.g., a virus, parasite or bacteria). In some embodiments a composition or pharmaceutical composition is substantially free of endotoxin. In some embodiments a composition or pharmaceutical composition is sterile. In certain embodiments, a composition or pharmaceutical composition comprises an immune-stimulating peptide and a suitable diluent (e.g., phosphate buffered saline).

The pharmaceutical compositions described herein may be configured for administration to a subject in any suitable form and/or amount according to the therapy in which they are employed. For example, a pharmaceutical composition configured for parenteral administration (e.g., by injection or infusion), may take the form of a suspension, solution or emulsion in an oily or aqueous vehicle and it may contain formulation agents, excipients, additives and/or diluents such as aqueous or non-aqueous solvents, co-solvents, suspending solutions, preservatives, stabilizing agents and or dispersing agents. In some embodiments a pharmaceutical composition suitable for parenteral administration may contain one or more excipients. In some embodiments a pharmaceutical composition is lyophilized to a dry powder form. In some embodiments a pharmaceutical composition is lyophilized to a dry powder form, which is suitable for reconstitution with a suitable pharmaceutical solvent (e.g., water, saline, an isotonic buffer solution (e.g., PBS), and the like). In certain embodiments, reconstituted forms of a lyophilized pharmaceutical composition are suitable for parenteral administration (e.g., intravenous administration) to a mammal.

In certain embodiments, a pharmaceutical composition is configured for oral administration and may be formulated as a tablet, microtablet, minitablets, micropellets, powders granules, capsules (e.g., capsules filled with microtablets, micropellets, powders or granules), emulsions or solutions. Pharmaceutical compositions configured for oral administration may comprise suitable coatings to delay or sustain release of the active ingredient (e.g., a binding agent), non-limiting examples of which include enteric coatings such as fatty acids, waxes, shellac, plastics, methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, cellulose acetate trimellitate, sodium alginate, zein, plant fibers, the like and combinations thereof.

In some embodiments a pharmaceutical composition described herein may be configured for topical administration and may include one or more of a binding and/or lubricating agent, polymeric glycols, gelatins, cocoa-butter or other suitable waxes or fats. In some embodiments a pharmaceutical composition described herein is incorporated into a topical formulation containing a topical carrier that is generally suited to topical drug administration and comprising any suitable material known to those skilled in the art. In certain embodiments, a topical formulation of a pharmaceutical composition is formulated for administration of a binding agent from a topical patch.

In certain embodiments, an optimal pharmaceutical composition will be determined by one skilled in the art depending upon, for example, the intended route of administration, delivery format and desired dosage (see e.g., Remington '95 or Remington 2013, supra). In certain embodiments, such compositions may influence the physical state, stability, rate of in vivo release and rate of in vivo clearance of the antibody drug conjugates of the invention. A pharmaceutical composition can be manufactured by any suitable manner, including, e.g., by means of conventional mixing, dissolving, granulating, dragee-making, levigating, emulsifying, encapsulating, entrapping or tableting processes (e.g., see methods described in Remington '95 or Remington 2013).

In some embodiments, presented herein is a composition or pharmaceutical composition for use as a medicament for the treatment of cancer or a neoplastic disorder in a subject, wherein the composition or pharmaceutical composition comprises an immune-stimulating peptide described herein. In some embodiments, presented herein is a composition or pharmaceutical composition comprising an immune-stimulating peptide described herein for use in the treatment of cancer or a neoplastic disorder.

Route

In some embodiments, an immune-stimulating peptide and a checkpoint inhibitor are administered to a subject by a suitable route of administration. In some embodiments, a composition described herein is administered to a subject by a suitable route of administration. A route of administration can be chosen by an individual care-giver or physician in view of a patient's condition. In some embodiments, a route of administration used herein is a route disclosed in, or taught in Fingl et al. 1975, in “The Pharmacological Basis of Therapeutics”, which is incorporated herein by reference in its entirety. Non-limiting examples of routes of administration include topical or local (e.g., transdermally or cutaneously, (e.g., on the skin or epidermis), in or on the eye, intranasally, transmucosally, in the ear, inside the ear (e.g., behind the ear drum), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranially, intraarticular, into a joint space, intracardiac (into the heart), intracavernous injection, intralesional (into a skin lesion), intraosseous infusion (into the bone marrow), intrathecal (into the spinal canal), intrauterine, intravaginal, intratumoral, intravesical infusion, intravitreal), the like or combinations thereof.

In some embodiments one or more immune-stimulating peptides, one or more checkpoint inhibitors, or a composition described herein is provided to a subject for self-administration or for administration to the subject by another (e.g., a care-giver). For example a composition described herein can be provided with an instruction written by a medical practitioner that authorizes a patient to be provided a composition or treatment described herein (e.g., a prescription). In another example, a composition can be provided to a subject wherein the subject self-administers a composition orally, intravenously, topically or by way of an inhaler, for example.

Formulations

One or more compositions (e.g., pharmaceutical compositions) can be formulated to be compatible with a particular route of administration or use. Compositions for parenteral, intradermal, or subcutaneous administration can include a sterile diluent, such as water, saline solution, fixed oils, polyethylene glycols, glycerin, propylene glycol or other synthetic solvents. A pharmaceutical composition may contain one or more preservatives to prevent microorganism growth (e.g., antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose). Antibacterial and antifungal agents include, for example, parabens, chlorobutanol, phenol, ascorbic acid and thimerosal.

In some embodiments, an immune-stimulating peptide, checkpoint inhibitor or combination thereof is formulated as a lyophilized powder. In some embodiments, a pharmaceutical composition described herein is formulated as a lyophilized powder. In certain embodiments, a lyophilized powder is formulated for reconstitution in a solvent that is suitable for injection into a mammal (e.g., a human). In certain embodiments, a lyophilized powder is formulated for oral administration, for example in the form of a granular powder or microparticles filled into capsules or compressed into tablets.

Compositions for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and polyethylene glycol), and suitable mixtures thereof. Fluidity can be maintained, for example, by the use of a coating such as lecithin, or by the use of surfactants. In some embodiments, a pharmaceutical composition includes an agent that delays absorption, for example, aluminum monostearate and gelatin which can prolong absorption of injectable compositions. In some embodiments, a pharmaceutical composition comprises polysorbate 20 or polysorbate 80, for example, up to 1%. Other non-limiting additives include histidine HCl, and α,α-trehalose dehydrate.

In some embodiments, one can administer compositions for use according to the methods of the invention in a local rather than systemic manner, for example, via direct application to the skin, mucous membrane or region of interest for treating, including using a depot or sustained release formulation.

In some embodiments, active ingredients (e.g., one or more immune-stimulating peptides, one or more checkpoint inhibitors) can be administered alone or formulated as a composition (e.g., a pharmaceutical composition). In other embodiments, one or more immune-stimulating peptides can be administered in combination with one or more additional materials (e.g., one or more chemotherapeutic agents or T-cell activators), for example, or as two separate compositions or as a single composition where the additional material(s) is (are) mixed or formulated together with one or more immune-stimulating peptides. For example, without being limited thereto, one or more immune-stimulating peptides can be formulated with additional excipients, or additional active ingredients.

The pharmaceutical compositions can be manufactured by any suitable manner, including, e.g., by means of conventional mixing, dissolving, granulating, dragee-making, levigating, emulsifying, encapsulating, entrapping or tableting processes. Pharmaceutical compositions comprising an immune-stimulating peptide and a checkpoint inhibitor described herein can be formulated in any suitable manner using one or more pharmaceutically acceptable carriers, non-limiting examples of which include carriers, solvents, salts, excipients, additives, preservatives, and/or auxiliaries. Proper formulation can depend upon the route of administration chosen. In particular, pharmaceutical compositions can comprise any suitable carrier, formulation, or ingredient, the like or combinations thereof as listed in “Remington: The Science And Practice Of Pharmacy” Mack Publishing Co., Easton, Pa., 19^{th} Edition, (1995)(hereafter, Remington '95), or “Remington: The Science And Practice Of Pharmacy”, Pharmaceutical Press, Easton, Pa., 22^{nd} Edition, (2013)(hereafter, Remington 2013), the contents of which are incorporated herein by reference in their entirety. The various materials listed herein, alone or in combination, can be incorporated into or used with the materials described in Remington '95 or Remington 2013. Any suitable techniques, carriers, and excipients can be used, including those understood in the art; e.g., as described in Remington '95 or Remington 2013.

In some embodiments, a pharmaceutical composition described herein can be formulated, for example, as a topical formulation. The topical formulation may include, for example, a formulation such as a gel formulation, a cream formulation, a lotion formulation, a paste formulation, an ointment formulation, an oil formulation, and a foam formulation. The composition further may include, for example, an absorption emollient.

A composition described herein can be administered on a daily basis, on an as-needed basis, or on a regular interval such as twice daily, three times daily, every other day, etc. A composition can be administered for a period of time ranging from a single as needed administration to administration for 1 day to multiple years, or any value there between, (e.g., 1 to 90 days, 1 to 60 days, 1 to 30 days, etc.). The dosages described herein can be daily dosages or the dosage of an individual administration, for example, even if multiple administrations occur (e.g., 2 sprays into a nostril).

In some embodiments, a composition described herein can be formulated for administration into the upper respiratory track/bronchi or nasal cavities. For example a composition can be formulated as an aerosol formulation, including formulated for use in a nebulizer or an inhaler. The compositions may include, for example, one or more of dichlorodifluoromethane, trichlorofluoromethane, dichlorotetrafluoroethane, carbon dioxide, and the like. The pharmaceutical compositions can be formulated for use in a nebulizer or an inhaler, for example.

A pharmaceutical composition may comprise one or more suitable carriers. In some embodiments, a carrier includes one or more chemical compounds that facilitate the incorporation of an active ingredient (e.g., one or more immune-stimulating peptides) into cells or tissues. For example, dimethyl sulfoxide (DMSO) is a commonly utilized carrier as it facilitates the uptake of many compounds and peptides into the cells or tissues of an organism. In some embodiments, a pharmaceutical carrier for a composition described herein can be selected from castor oil, ethylene glycol, monobutyl ether, diethylene glycol monoethyl ether, corn oil, dimethyl sulfoxide, ethylene glycol, isopropanol, soybean oil, glycerin, zinc oxide, titanium dioxide, glycerin, butylene glycol, cetyl alcohol, and sodium hyaluronate.

In certain embodiments, a pharmaceutical composition comprises hydrophobic excipients, additives, or other hydrophobic components. A pharmaceutical carrier for certain hydrophobic peptides can be a co-solvent system comprising benzyl alcohol, a nonpolar surfactant, a water-miscible organic polymer, and an aqueous phase. A common co-solvent system contemplated for use herein is the VPD co-solvent system, which is a solution of 3% w/v benzyl alcohol, 8% w/v of the nonpolar surfactant POLYSORBATE 80™, and 65% w/v polyethylene glycol 300, made up to volume in absolute ethanol. Naturally, the proportions of a co-solvent system can be varied considerably without destroying its solubility and toxicity characteristics. Furthermore, the identity of the co-solvent components can be varied: for example, other low-toxicity nonpolar surfactants can be used instead of POLYSORBATE 80™; the fraction size of polyethylene glycol can be varied; other biocompatible polymers can replace polyethylene glycol, e.g., polyvinyl pyrrolidone; and other sugars or polysaccharides can substitute for dextrose.

Alternatively or additionally, other carriers can be employed, if required. Liposomes and emulsions are well known examples of delivery vehicles or carriers for hydrophobic drugs and drug compositions. Additionally, a composition described herein can be delivered using a sustained-release system, such as semipermeable matrices of solid hydrophobic polymers containing the therapeutic agent. The pharmaceutical compositions described herein can be administered to a patient per se, or in pharmaceutical compositions where they are mixed with other active ingredients, as in combination therapy, or suitable carriers or excipient(s). The compounds and compositions can be formulated with salts or excipients, such as for example, sodium or meglumine. Techniques for formulation and administration can be found in “Remington's Pharmaceutical Sciences,” Mack Publishing Co., Easton, Pa., 18th edition, 1990.

Furthermore, the compounds and compositions used herein can be stable over an extended period of time, for example on the order of months or years. Compositions described herein, in some embodiments, may comprise a preservative. The preservative can comprise a quaternary ammonium compound, such as benzalkonium chloride, benzoxonium chloride, benzethonium chloride, cetrimide, sepazonium chloride, cetylpyridinium chloride, or domiphen bromide (BRADOSOL®). The preservative can comprise an alkyl-mercury salt of thiosalicylic acid, such as thiomersal, phenylmercuric nitrate, phenylmercuric acetate or phenylmercuric borate. The preservative can comprise parabens, such as methylparaben or propylparaben. The preservative can comprise an alcohol, such as chlorobutanol, benzyl alcohol or phenyl ethyl alcohol. The preservative can comprise a biguanide derivative, such as chlorohexidine or polyhexamethylene biguanide. The preservative can comprise sodium perborate, imidazolidinyl urea, and/or sorbic acid. The preservative can comprise stabilized oxychloro complexes, such as known and commercially available under the trade name PURITE®). The preservative can comprise polyglycol-polyamine condensation resins, such as known and commercially available under the trade name POLYQUART® from Henkel KGaA. The preservative can comprise stabilized hydrogen peroxide generated from a source of hydrogen peroxide for providing an effective trace amount of resultant hydrogen peroxide, such as sodium perborate tetrahydrate. The preservative can be benzalkonium chloride.

The preservative can enable a composition to be used on multiple occasions. The preservative can reduce the effects of one or more of acid exposure, base exposure, air exposure, heat, and light on the active ingredient. The compounds and pharmaceutical compositions described herein can include any suitable buffers, such as for example, sodium citrate buffer and/or sequestering agents, such as edetate disodium sequestering agent. Ingredients, such as meglumine, may be added to adjust the pH of a composition or compound described herein. Compounds and compositions described herein may comprise sodium and/or iodine, such as organically bound iodine. Compositions and compounds used herein may be provided in a container in which the air is replaced by another substance, such as nitrogen.

Certain embodiments provide compositions comprising one or more active ingredients (e.g., an immune-stimulating peptide, a checkpoint inhibitor, etc.) in an amount effective to achieve its intended purpose (e.g., a therapeutically effective amount). In some embodiments, an “effective amount” is an amount that can induce mitosis, growth and/or proliferation of T-cells. A “therapeutically effective amount” means an amount to prevent or treat a cancer. In certain embodiments, a “therapeutically effective amount” is an amount sufficient to block, inhibit, ameliorate, abrogate, or suppress growth, metastasis or viability of a cancer (e.g., cancer cells, e.g., a tumor). In certain embodiments, a “therapeutically effective amount” is an amount sufficient to induce necrosis, death or apoptosis of one or more cancer cells in a subject. In certain embodiments, a “therapeutically effective amount” is an amount sufficient to inhibit, reduce the severity of, delay the onset of, suppress, ameliorate, or abrogate one or more symptoms associate with a cancer. In certain embodiments, a “therapeutically effective amount” is an amount sufficient to prolong or sustain life, or improve the quality of life of a subject having a cancer, compared to the length or quality of life that the subject would have in the absence of a cancer treatment. Determination of a therapeutically effective amount is well within the capability of those skilled in the art (e.g., a medical practitioner), especially in light of the detailed disclosure provided herein.

In some embodiments, a therapeutically effective amount is an amount needed for a significant quantity of a pharmaceutical composition (or immune-stimulating peptide therein) to contact a desired region or tissue where prevention or treatment of a cancer is desired.

The overall beneficial effect of a treatment described herein can be determined by comparing the condition or disease state of a subject who received a treatment described herein to one or more individuals who have not received treatment, or to the same patient prior to, or after cessation of a treatment. A beneficial effect of a treatment may be complete (no detectable symptoms or cancer) or partial, such that fewer symptoms or amounts of a cancer are observed than would likely occur absent treatment. Accordingly, in some embodiments, a treatment disclosed herein provides for a complete or a partial elimination of a cancer in a subject.

Dose

In certain embodiments, an immune-stimulating peptide and/or a checkpoint inhibitor is administered at a suitable therapeutically effective amount or a dose (e.g., at a suitable volume and concentration, which sometimes depends, in part, on a particular route of administration). In some embodiments, a therapeutically effective amount of an immune-stimulating peptide and/or a checkpoint inhibitor is an amount needed to obtain an effective therapeutic outcome. In certain embodiments, a therapeutically effective amount of immune-stimulating peptide and/or a checkpoint inhibitor is an amount sufficient to prevent, treat, reduce the severity of, delay the onset of, and/or alleviate a symptom of a neoplastic disorder or cancer, as contemplated herein. In certain embodiments, a therapeutically effective amount of an immune-stimulating peptide and/or a checkpoint inhibitor is an amount sufficient to block, inhibit, ameliorate, abrogate, or suppress growth, viability and/or metastasis of a cancer or cancer cell. In certain embodiments, a therapeutically effective amount of an immune-stimulating peptide and/or a checkpoint inhibitor is an amount sufficient to induce death, necrosis or apoptosis of a cancer or cancer cell. In certain embodiments, a therapeutically effective amount of immune-stimulating peptide and/or a checkpoint inhibitor is an amount sufficient to decrease, inhibit, or reduce mitosis of a cancer cell.

In certain embodiments, a therapeutically effective amount of an immune-stimulating peptide and/or a checkpoint inhibitor is independently selected from an amount of 0.01 mg/kg (e.g., per kg body-weight of a subject) to 500 mg/kg, 0.1 mg/kg to 500 mg/kg, 0.1 mg/kg to 400 mg/kg, 0.01 mg/kg to 300 mg/kg, 0.1 mg/kg to 500 mg/kg, 0.1 mg/kg to 300 mg/kg, 0.1 mg/kg to 200 mg/kg, 0.1 mg/kg to 150 mg/kg, 0.1 mg/kg to 100 mg/kg, 0.1 mg/kg to 75 mg/kg, 0.1 mg/kg to 50 mg/kg, 0.1 mg/kg to 25 mg/kg, 0.1 mg/kg to 10 mg/kg, 0.1 mg/kg to 5 mg/kg or 0.1 mg/kg to 1 mg/kg. In certain embodiments a therapeutically effective amount of an immune-stimulating peptide and/or a checkpoint inhibitor is at least 0.01 mg/kg, at least 0.1 mg/kg, at least 1 mg/kg, at least 10 mg/kg, or at least 50 mg/kg. In some aspects a therapeutically effective amount of an immune-stimulating peptide and/or a checkpoint inhibitor is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg. Volumes suitable for various routes of administration are known in the art. For example, 0.1 ml-100 ml of a composition can be safely administered intravenously to an adult human subject.

In certain embodiments, administering a therapeutically effective amount of an immune-stimulating peptide and/or a checkpoint inhibitor comprises one or more independent administrations. For example, in some embodiments, administering a therapeutically effective amount of an immune-stimulating peptide and/or a checkpoint inhibitor comprises administering a suitable dose daily, every other day, every third day, once weekly, twice weekly, or three times weekly. In some embodiments, administering a therapeutically effective amount of an immune-stimulating peptide and/or a checkpoint inhibitor comprises administering a suitable dose every hour, every 2 hours, every 4 hours, every 6 hours, once a day, twice a day, three times a day, four times a day or more. In some embodiments, administering a therapeutically effective amount of an immune-stimulating peptide and/or a checkpoint inhibitor comprises administering a constant dose for example, by intravenous administration, over a period of one or more hour, one or more days or one or more weeks. An immune-stimulating peptide and/or a checkpoint inhibitor may be administered at different times, at different doses and at different frequency to obtain an optimal therapeutically effective amount of the combination treatment.

Kits

In some embodiments the compositions, formulations, combination products and materials described herein can be included as part of kits, which kits can include one or more of the compositions described herein, formulations of the same, chemotherapeutic agents for combination treatments and products and other materials described herein. In some embodiments the kit comprises one or more immune-stimulating peptides, or a pharmaceutical composition comprising the same. In some embodiments a kit comprises one or more immune-stimulating peptides and one or more checkpoint inhibitors. In some embodiments, a kit comprises an immune-stimulating peptide comprising a label. In some embodiments a kit comprises a chemotherapeutic agent. In some embodiments the products, compositions, kits, formulations, etc. can come in an amount, package, and/or product format with enough medication to treat a patient for 1 day to 1 year, 1 day to 180 days, 1 day to 120 days, 1 day to 90 days, 1 day to 60 days, 1 day to 30 days, or any day or number of days there between. A composition in a kit often includes materials for convenient administration. For example, where a composition is to be administered intravenously, the kit may include a suitable sterile syringe and sterile needle.

In some embodiments, a kit comprises suitable packaging materials. A kit optionally includes a label or packaging insert including a description of the components or instructions for use in vitro, in vivo, or ex vivo, of the components therein. Exemplary instructions include instructions for a method, treatment protocol or therapeutic regimen described herein. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer-readable medium, optical disk such as CD- or DVD-ROM/RAM, DVD, MP3, magnetic tape, or an electrical storage media such as RAM and ROM or hybrids of these such as magnetic/optical storage media, FLASH media or memory type cards. Labels or inserts can include identifying information of one or more components therein, dose amounts, clinical pharmacology of the active ingredient(s) including mechanism of action, pharmacokinetics (PK) and pharmacodynamics (PD). Labels or inserts can include information identifying manufacturer information, lot numbers, manufacturer location and date.

Labels or inserts can include information on a condition, cancer, disorder, disease or symptom for which a kit component may be used. Labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. Kits of the invention therefore can additionally include labels or instructions for practicing any of the methods and uses of the invention described herein.

Labels or inserts can include information on any benefit that a component may provide, such as a prophylactic or therapeutic benefit. Labels or inserts can include information on potential adverse side effects, such as warnings to the subject or clinician regarding situations where it would not be appropriate to use a particular composition. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Components of the kit can be enclosed within an individual container and all of the various containers can be within a single package. Invention kits can be designed for cold storage.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed.

### EXAMPLES

### Example 1

INT-151 is a drug formulation comprising the dipeptide tyrosyl-glycine. The dipeptide tyrosyl-glycine, is the active component of a leukocyte-derived dialysate that augments and accelerates the delayed-type hypersensitivity response to recall antigens. In a double blind, randomized, placebo-controlled trial, the tyrosyl-glycine containing dialysate, was administered intradermally every other week, was proven to be safe and was shown to slow the occurrence of AIDS-defining events, compared to placebo, in symptomatic patients with HIV disease. In addition, in a mouse myeloma model, treatment with the tyrosyl-glycine containing dialysate resulted in enhanced survival compared to placebo control. Without being limited to theory, the mechanisms responsible for the effects of tyrosyl-glycine may be, in part, due to its ability to enhance interferon-gamma, enhance expression of receptors for IL-2, increase natural killer cells, increase CD4+ T cells, alter calcium uptake by peripheral blood monocytes, and alter proliferation of certain cell types in response to mitogens.

In an effort to determine if INT-151 would be effective as an anti-cancer agent when administered in combination with a checkpoint inhibitor, a syngeneic mouse model of lymphoma (E.G7-OVA) was used.

Treatment

Treatment was initiated six days after the s.c. cell inoculation into the flank in six groups of C57BL/6 mice (n=10) according to a standard E.G7-OVA-e214 experimental protocol. All test article doses were administered i.p. in a fixed volume as described above. Dosing was not adjusted for body-weight. Tumors were initiated in four groups of mice with E.G7-OVA murine lymphoma cells.
Group 1 received vehicle (saline) weekly to study end, starting on Day 6 (every week to end, start on Day 6).
Group 2 received anti-CTLA-4 at a dose of 100 μg/animal on Day 6 followed by anti-CTLA-4 at a dose of 50 μg/animal on Days 9 and 12.
Group 3 received INT-151 at a dose of 100 μg/animal every week to end, start on Day 6.
Group 4 received INT-151 at a dose of 100 μg/animal every week to end, start on Day 6 in combination with anti-CTLA-4 monotherapy as described above.

Animals were monitored daily for overall health and well-being. In addition, animal weight and tumor volume were measured weekly. Animals were followed for survival until 6 weeks after tumor implantation, and were euthanized when pre-defined morbidity parameters were reached. Animals were monitored for signs of toxicity by frequent observation and by regular body-weight measurements over the course of the study. All groups showed body-weight increases.

Materials and Methods

Forty female C57BL/6 mice were between 8 and 12 weeks old on the start date. The animals were fed ad libitum water (reverse osmosis, 1 ppm Cl) and NIH 31 Modified and Irradiated LabDiet® consisting of 18.0% crude protein, 5.0% crude fat, and 5.0% crude fiber. The mice were housed on irradiated Enrich-o'Cobs Laboratory Animal Bedding in static microisolators on a 12-hour light cycle at 21-22° C. (70-72° F.) and at 40 to 60% humidity. The protocol complies with the recommendations of the Guide for Care and Use of Laboratory Animals with respect to restraint, husbandry, surgical procedures, feed and fluid regulation, and veterinary care.

Tumors were initiated with E.G7-OVA murine lymphoma cells. E.G7-OVA cells were cultured in RPMI-1640 Medium supplemented with 10% fetal bovine serum, 2 mM glutamine, 10 mM HEPES, 0.4 mg/mL G418, 4.5 g/L glucose, 0.075% sodium bicarbonate, 50 μM β-mercaptoethanol, 1 mM sodium pyruvate, 100 units/mL sodium penicillin G, 100 μg/mL streptomycin sulfate and 25 μg/mL gentamicin. The cells were grown in tissue culture flasks in a humidified incubator at 37° C., in an atmosphere of 5% CO2 and 95% air.

Test mice were randomized into four groups (n=10) based on Day 1 bodyweight. E.G7-OVA cells used for implantation were harvested during log phase growth and resuspended in cold phosphate buffered saline (PBS) at 1×10⁷ cells/mL. Each mouse was injected subcutaneously in the right flank with 1×10⁶ E.G7-OVA cells (0.1 mL cell suspension) on Day 1 of the study.

Animals were monitored individually. The endpoint of the experiment was a tumor volume of 2000 mm³ or 47 days, whichever comes first. Responders can be followed longer. When the endpoint is reached, the animals were euthanized.

INT-151 was synthesized using standard peptide synthesis chemistry and confirmed to consist of the dipeptide Tyr-Gly, with molecular weight 238.51 confirmed. Purity was >98% by HPLC. INT-151 preparations were prepared weekly by dissolving 1 mg of the peptide into sterile saline (vehicle) for a 1 mg/mL stock solution.

The monoclonal antibody anti-CTLA-4 (clone 9H10) was purchased from BioXcell. All agents were prepared according to protocol instructions. All antibody dosing solutions were prepared fresh daily and stored at 4 ° C.

Anti-CTLA-4 antibody dosing solution was prepared for each dose by diluting an aliquot of the stock (6.15 mg/mL) to 0.5 mg/mL in sterile PBS to provide a 100 μg/animal dose in a fixed volume of 0.2 mL. An aliquot of the 0.5 mg/mL dosing solution was further diluted in sterile PBS to provide a 50 μg/animal dose in a fixed volume of 0.2 mL.

Results

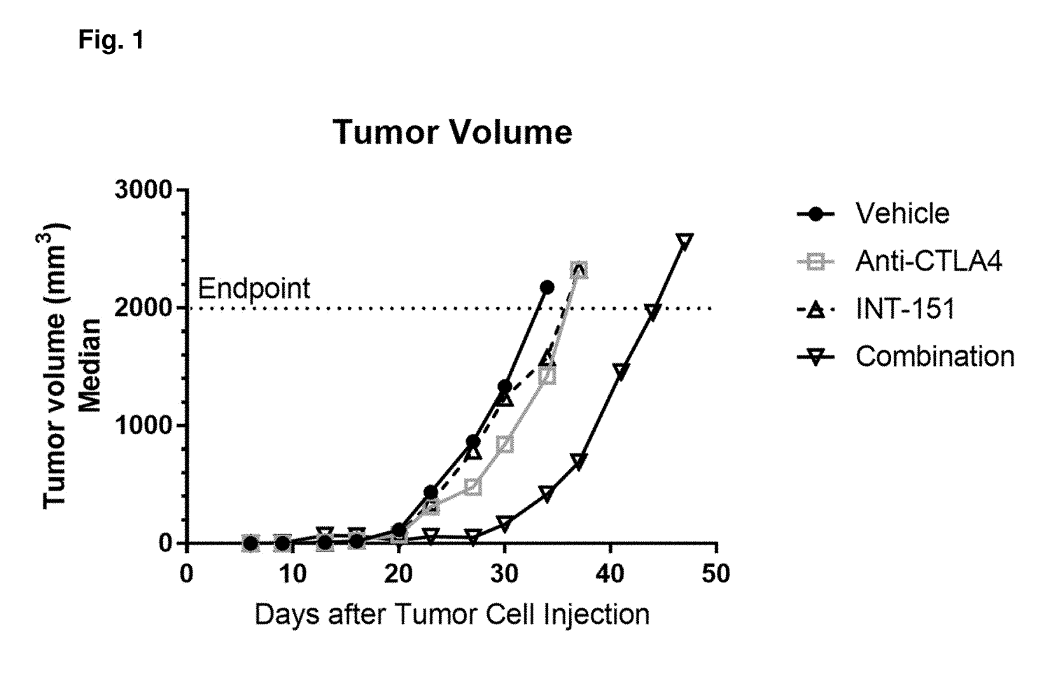

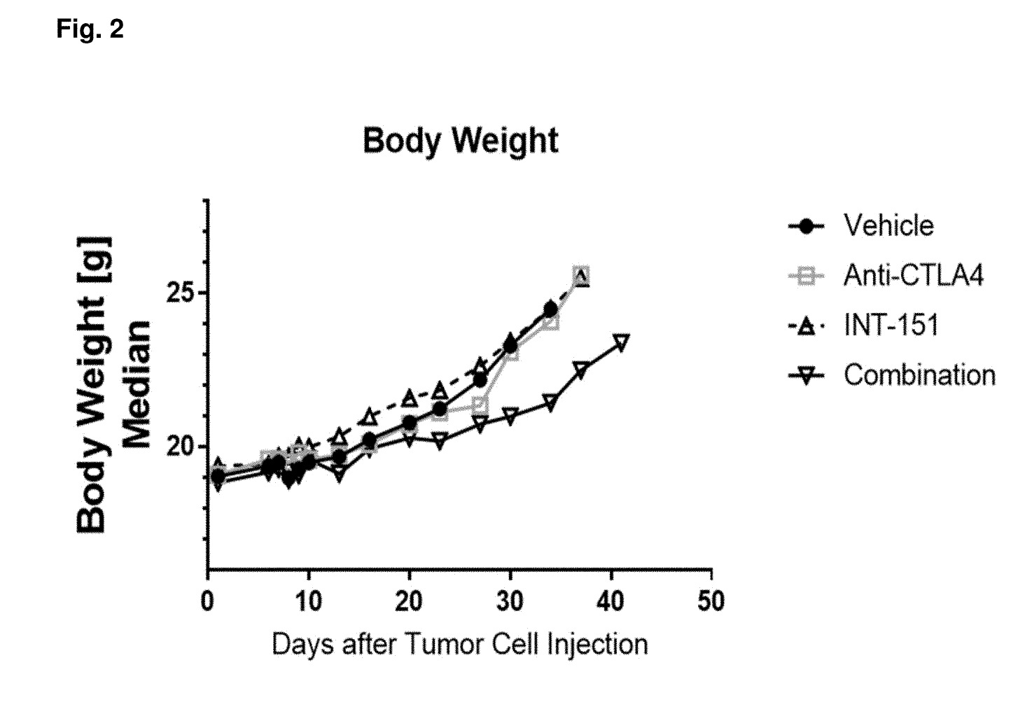

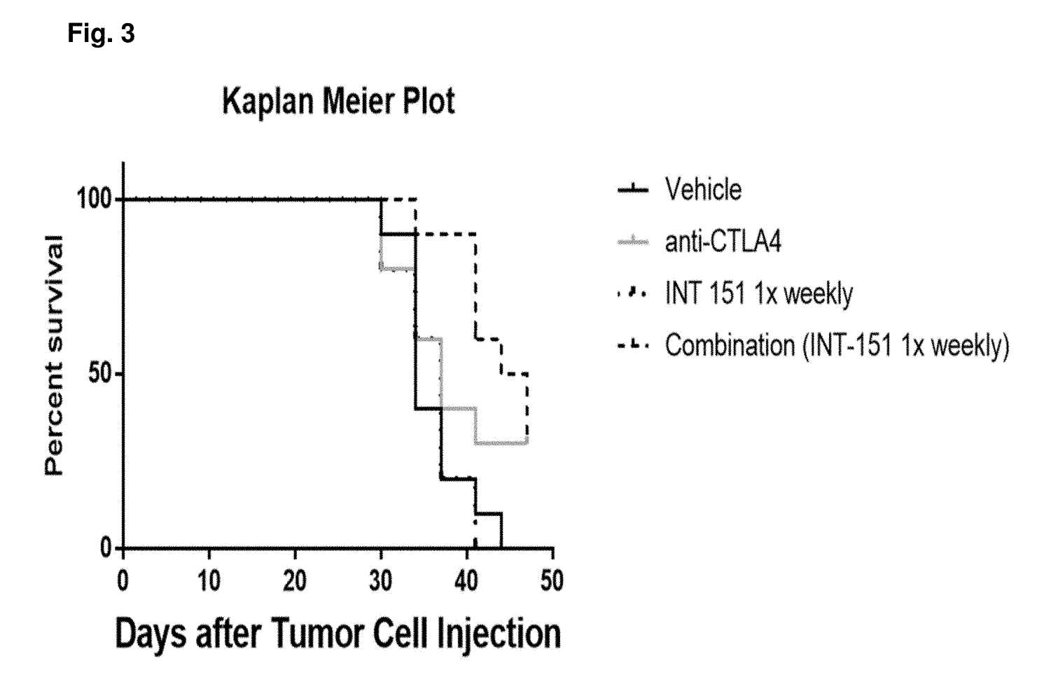

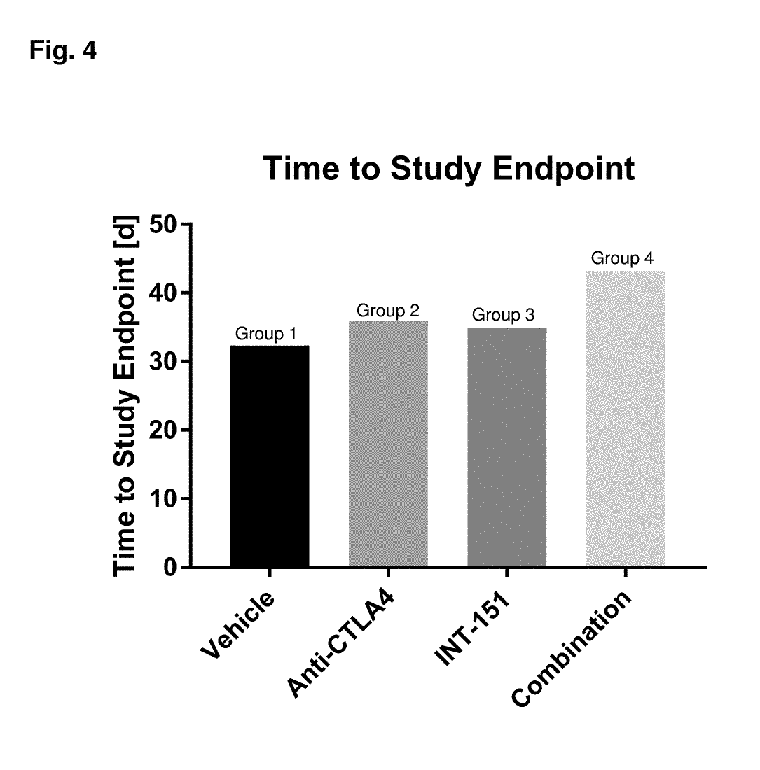

Compared to untreated animals in Group 1, animals treated with either INT-151 or anti-CTLA-4 alone had slight positive clinical improvement in median tumor volume growth (FIG. 1) and body-weight (FIG. 2). Animals treated with anti-CTLA-4 alone, but not INT-151, demonstrated improved overall survival compared to control (FIG. 3), but no prolonged survival in animals that would ultimately die. In contrast to untreated animals or animals treated with either therapy alone, mice which received the combination of INT-151 and CTLA-4 demonstrated marked clinical improvement evidenced by reduced tumor size and enhanced survival (FIG. 4).

Conclusions

INT-151 acts synergistically with anti-CTLA-4 to improve survival in a syngeneic mouse lymphoma model. These results strongly suggest that INT-151 can exert synergistic anti-cancer effects when used with anti-CTLA-4 against other cancer types, and when used with other checkpoint inhibitors or immunotherapies.

In this study, the synergistic effect of tyrosyl-glycine dipeptide (INT-151) on tumor growth and survival was demonstrated in a syngeneic mouse model of lymphoma. The synergistic effect was evidenced on all parameters measured, including weight gain, tumor volume, time to study endpoint, and survival.

### Example 2

### Pilot Dose Response Study

In this study a once-weekly dose of INT-151 was compared to a 3× weekly dose as a single agent and in combination with anti-CTLA-4. Treatment was initiated six days after the s.c. cell inoculation into the flank in six groups of C57BL/6 mice (n=10) according to the E.G7-OVA-e215 protocol. All test article doses were administered i.p. in a fixed volume as described above. Dosing was not adjusted for body-weight. Tumors were initiated in four groups of mice with E.G7-OVA murine lymphoma cells.
Group 1 received vehicle (saline) weekly to study end, starting on Day 6 (every week to end, start on Day 6).
Group 2 received anti-CTLA-4 at a dose of 100 μg/animal on Day 6 followed by anti-CTLA-4 at a dose of 50 μg/animal on Days 9 and 12.
Group 3 received INT-151 at a dose of 100 μg/animal every week to end, start on Day 6.
Group 4 received INT-151 at a dose of 100 μg/animal 3 times a week to end, start on Day 6.
Group 5 received INT-151 at a dose of 100 μg/animal every week to end, start on Day 6 in combination with anti-CTLA-4 therapy as described above.
Group 6 received INT-151 at a dose of 100 μg/animal 3 times a week to end, start on Day 6 in combination with anti-CTLA-4 therapy as described above.

Animals were monitored daily for overall health and well-being. In addition, animal weight and tumor volume were measured weekly. Animals were followed for survival until 6 weeks after tumor implantation, and were euthanized when pre-defined morbidity parameters were reached. Animals were monitored for signs of toxicity by frequent observation and by regular body-weight measurements over the course of the study. All groups showed body-weight increases.

Materials and Methods

Sixty female C57BL/6 mice were between 8 and 12 weeks old on the start date. The animals were fed ad libitum water (reverse osmosis, 1 ppm Cl) and NIH 31 Modified and Irradiated Lab Diet® consisting of 18.0% crude protein, 5.0% crude fat, and 5.0% crude fiber. The mice were housed on irradiated Enrich-o'Cobs Laboratory Animal Bedding in static microisolators on a 12-hour light cycle at 21-22° C. (70-72° F.) and at 40-60% humidity. Charles River Discovery Services North Carolina (CR Discovery Services) specifically complies with the recommendations of the Guide for Care and Use of Laboratory Animals with respect to restraint, husbandry, surgical procedures, feed and fluid regulation, and veterinary care. The animal care and use program at CR Discovery Services is accredited by the Association for Assessment and Accreditation of Laboratory Animal Care International, which assures compliance with accepted standards for the care and use of laboratory animals.

Tumors were initiated with E.G7-OVA murine lymphoma cells. E.G7-OVA cells were cultured in RPMI-1640 Medium supplemented with 10% fetal bovine serum, 2 mM glutamine, 10 mM HEPES, 0.4 mg/mL G418, 4.5 g/L glucose, 0.075% sodium bicarbonate, 50 μM β-mercaptoethanol, 1 mM sodium pyruvate, 100 units/mL sodium penicillin G, 100 μg/mL streptomycin sulfate and 25 μg/mL gentamicin. The cells were grown in tissue culture flasks in a humidified incubator at 37° C., in an atmosphere of 5% CO2 and 95% air.

Test mice were randomized into six groups (n=10) on Day 1 based on bodyweight. E.G7-OVA cells used for implantation were harvested during log phase growth and resuspended in cold phosphate buffered saline (PBS) at 1×10⁷ cells/mL. Each mouse was injected subcutaneously in the right flank with 1×10⁶ E.G7-OVA cells (0.1 mL cell suspension) on Day 1 of the study.

Animals were monitored individually. The endpoint of the experiment was a tumor volume of 2000 mm³ or 45 days, whichever came first. When the endpoint was reached, the animals were euthanized.

INT-151 was synthesized using standard peptide synthesis chemistry and confirmed to consist of the dipeptide Tyr-Gly, with molecular weight 238.51 confirmed. Purity was >98% by HPLC. INT-151 preparations were prepared weekly by dissolving 1 mg of the peptide into sterile saline (vehicle) for a 1 mg/mL stock solution.

The monoclonal antibody anti-CTLA-4 (clone 9H10) was purchased from BioXcell. All agents were prepared according to protocol instructions. All antibody dosing solutions were prepared fresh daily and stored at 4° C.

Anti-CTLA-4 antibody dosing solution was prepared for each dose by diluting an aliquot of the stock (6.15 mg/mL) to 0.5 mg/mL in sterile PBS to provide a 100 μg/animal dose in a fixed volume of 0.2 mL. An aliquot of the 0.5 mg/mL dosing solution was further diluted in sterile PBS to provide a 50 μg/animal dose in a fixed volume of 0.2 mL.

Results

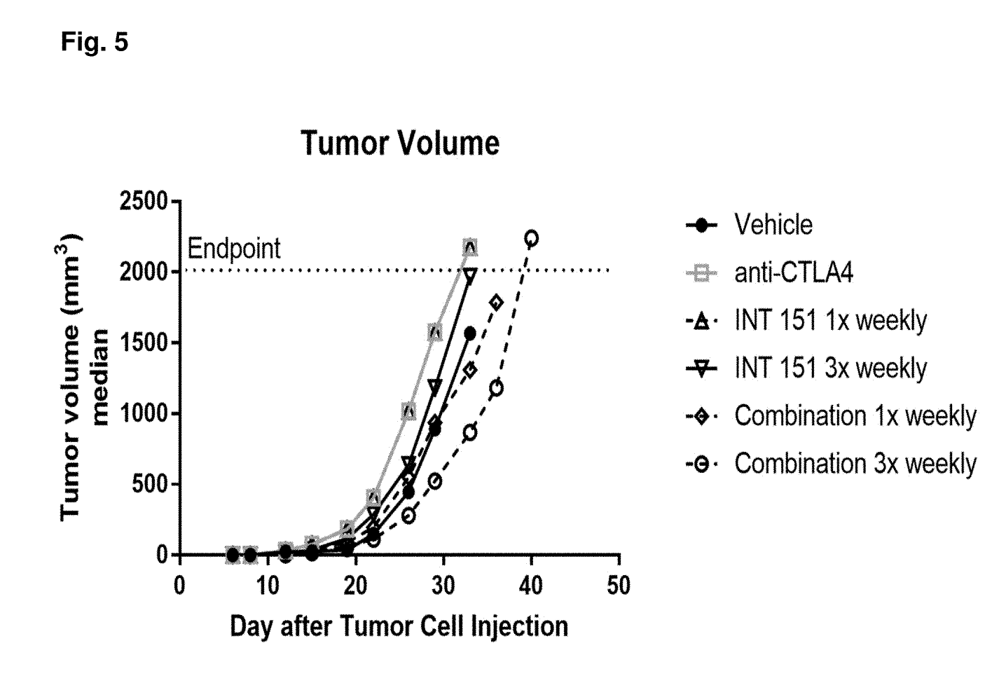

Compared to untreated animals in Group 1, animals treated with 3 times weekly INT-151 as single agent had greater clinical improvement compared to the once weekly treatment. The combination of INT-151 administered 3x weekly and anti-CTLA-4 had the most clinical improvement among all treatment groups in median tumor volume and survival (e.g., see FIG. 5 and Table 1). Survival on day 40 was greatest in the INT-151 3× and anti-CTLA-4 combination with 55% whereas survival in the groups treated with the combination 1× INT-151 and anti-CTLA-4 was 30% each. No survivors were observed on day 40 in the anti-CTLA-4 or the INT-151 once weekly single treatment groups. Single agent INT-151 administered 3 times weekly resulted in greater survival (100-10%) than single agents anti-CTLA-4 (75 -0%) or INT-151 once weekly (60-0%) between day 33 and 40 of the study. The survival improvement lasted over a time period of 7 days (day 33-40). The results are summarized in Table 1.

TABLE 1
Survival in individual treatment groups comparing once and three times weekly
INT-151 alone and in combination with anti-CTLA-4

| | | Start # | Tumor | % Alive | % Alive | % Alive | % Alive | % Alive | % Alive |
|---|---|---|---|---|---|---|---|---|---|
| Group | Treatments | animals | take | on day 26 | on day 29 | on day 33 | on day 36 | on day 40 | on day 43 |
| 1 | vehicle | 10 | 10 | 80 | 60 | 10 | 10 | 10 | 10 |
| 2 | Anti CTLA-4 | 10 | 8 | 100 | 100 | 75 | 50 | 0 | 0 |
| 3 | INT-151 | 10 | 10 | 100 | 100 | 60 | 20 | 0 | 0 |
| | 1× weekly | | | | | | | | |
| 4 | INT-151 | 10 | 10 | 100 | 100 | 80 | 40 | 30 | 0 |
| | 3× weekly | | | | | | | | |
| 5 | INT-151 1× | 10 | 10 | 100 | 100 | 80 | 60 | 30 | 20 |
| | Anti CTLA-4 | | | | | | | | |
| | days 6, 9, 12 | | | | | | | | |
| 6 | INT-151 3× | 10 | 9 | 100 | 100 | 100 | 88 | 55 | 11 |
| | Anti CTLA-4 | | | | | | | | |

*Only animals with tumor take included in this section

INT-151 was well tolerated in each treatment group and in combination with anti-CTLA-4.

Conclusions

These results indicate biological activity of the single agent INT-151.

INT-151 synergizes with anti-CTLA-4 and improved survival in a syngeneic mouse lymphoma model. These results suggest that INT-151 may exert similar synergistic effects when used with anti-CTLA-4 in other cancer types, and/or with other checkpoint inhibitors or immunotherapies.

In this study, the synergistic effect of tyrosyl-glycine dipeptide (INT-151) on tumor growth and survival was demonstrated in a syngeneic mouse model of lymphoma. The synergistic effect was evidenced on all parameters measured, including weight gain, tumor volume, time to study endpoint, and survival.

### Effect of INT-151 Alone and in Combination with Anti-CTLA-4 on the Immune Cell Profile in Tumors and Peripheral Blood in Lymphoma Bearing Mice

In this study the immune cell profile in tumors and blood in lymphoma bearing mice was compared for a 3 times weekly dose of INT-151 and anti-CTLA-4 alone to the combination of 3 times weekly INT-151 plus anti-CTLA-4. Treatment was initiated in mice with established tumors (30-60 mm³) after the s.c. cell inoculation into the flank in six groups of C57BL/6 mice (n=6) according to the E.G7-OVA-e217 protocol. All test article doses were administered i.p. in a fixed volume as described above. Dosing was not adjusted for body-weight.
Group 1 received vehicle (saline) weekly to study end, starting on Day 1 after randomization (every week to end, day 17).
Group 2 received anti-CTLA-4 at a dose of 100 μg/animal on Day 6 followed by anti-CTLA-4 at a dose of 50 μg/animal on Days 9 and 12.
Group 3 received INT-151 at a dose of 100 μg/animal 3 times a week to end, start on Day 1
Group 4 received INT-151 at a dose of 100 μg/animal 3 times a week to end, start on Day 1 in combination with anti-CTLA-4 therapy as described above.

On day 17 after treatment began, when tumors reached a volume of 500-600 mm³, mice were sacrificed and tumors and blood was collected for Fluorescent Activated Cell Sorting (FACS) analysis to determine the following immune cell types as listed in the table below:

TABLE 2
CD4, CD8, Treg, granulocytic and monocytic MDSC

| Cell | | Antibody |
|---|---|---|
| population | Phenotypic Markers | panel |
| CD4 | CD45⁺CD3⁺CD4⁺CD8⁻ | CD45, CD3, CD4, CD8, |
| CD8 | CD45⁺CD3⁺CD4⁻CD8⁺ | CD11b, CD25, |
| Tregs | CD45⁺CD3⁺CD4⁺CD25⁺FoxP3⁺ | Ly-6G, Ly-6C, |
| granulocytic | CD45⁺CD3⁻ | FoxP3, LIVE/DEAD |
| MDSC | CD11b⁺Ly6G⁺Ly6C^{low} | |
| monocytic | CD45⁺CD3⁻CD11b⁺Ly6G⁻ | |
| MDSC | Ly6C^{high} | |

FoxP3, internal marker

TABLE 3
M1 and M2 Macrophage

| | | Antibody |
|---|---|---|
| Cell Population | Phenotypic Markers | Panel |
| M1 Macrophage | CD45⁺F4/80⁺Gr1⁻ | CD45, *CD206, CD11b, |
| | CD11b⁺CD206⁻ | Gr1, F4/80, LIVE/DEAD |
| M2 Macrophage | CD45⁺F4/80⁺ Gr1⁻ | |
| | CD11b⁺CD206⁺ | |

*CD206 internal marker

Animals were monitored daily for overall health and well-being. In addition, animal weight and tumor volume were measured weekly. Animals were monitored for signs of toxicity by frequent observation and by regular body-weight measurements over the course of the study. All groups showed body-weight increases.

Materials and Methods

Sixty female C57BL/6 mice were between 8 and 12 weeks old on the start date. The animals were fed ad libitum water (reverse osmosis, 1 ppm Cl) and NIH 31 Modified and Irradiated Lab Diet® consisting of 18.0% crude protein, 5.0% crude fat, and 5.0% crude fiber. The mice were housed on irradiated Enrich-o'Cobs Laboratory Animal Bedding in static microisolators on a 12-hour light cycle at 21-22° C. (70-72° F.) and at 40-60% humidity. Charles River Discovery Services North Carolina (CR Discovery Services) specifically complies with the recommendations of the Guide for Care and Use of Laboratory Animals with respect to restraint, husbandry, surgical procedures, feed and fluid regulation, and veterinary care. The animal care and use program at CR Discovery Services is accredited by the Association for Assessment and Accreditation of Laboratory Animal Care International, which assures compliance with accepted standards for the care and use of laboratory animals.

Tumors were initiated with E.G7-OVA murine lymphoma cells. E.G7-OVA cells were cultured in RPMI-1640 Medium supplemented with 10% fetal bovine serum, 2 mM glutamine, 10 mM HEPES, 0.4 mg/mL G418, 4.5 g/L glucose, 0.075% sodium bicarbonate, 50 μM β-mercaptoethanol, 1 mM sodium pyruvate, 100 units/mL sodium penicillin G, 100 μg/mL streptomycin sulfate and 25 μg/mL gentamicin. The cells were grown in tissue culture flasks in a humidified incubator at 37° C., in an atmosphere of 5% CO2 and 95% air.

E.G7-OVA cells used for implantation were harvested during log phase growth and resuspended in cold phosphate buffered saline (PBS) at 1×10⁷ cells/mL. Each mouse was injected subcutaneously in the right flank with 1×10⁶ E.G7-OVA cells (0.1 mL cell suspension) on Day 1 of the study.

Animals were to be monitored individually. Randomization into treatment groups was conducted when tumors reached an average of 43 mm³ (n=6). Mice were euthanized after 17 days to collect tumors and blood for FACS analysis.

INT-151 was synthesized using standard peptide synthesis chemistry and confirmed to consist of the dipeptide Tyr-Gly, with molecular weight 238.51 confirmed. Purity was >98% by HPLC. INT 151 preparations were prepared weekly by dissolving 1 mg of the peptide into sterile saline (vehicle) for a 1 mg/mL stock solution.

The monoclonal antibody anti-CTLA-4 (clone 9H10) was purchased from BioXcell. All agents were prepared according to protocol instructions. All antibody dosing solutions were prepared fresh daily and stored at 4° C.

Anti-CTLA-4 antibody dosing solution was prepared for each dose by diluting an aliquot of the stock (6.15 mg/mL) to 0.5 mg/mL in sterile PBS to provide a 100 μg/animal dose in a fixed volume of 0.2 mL. An aliquot of the 0.5 mg/mL dosing solution was further diluted in sterile PBS to provide a 50 μg/animal dose in a fixed volume of 0.2 mL.

Results

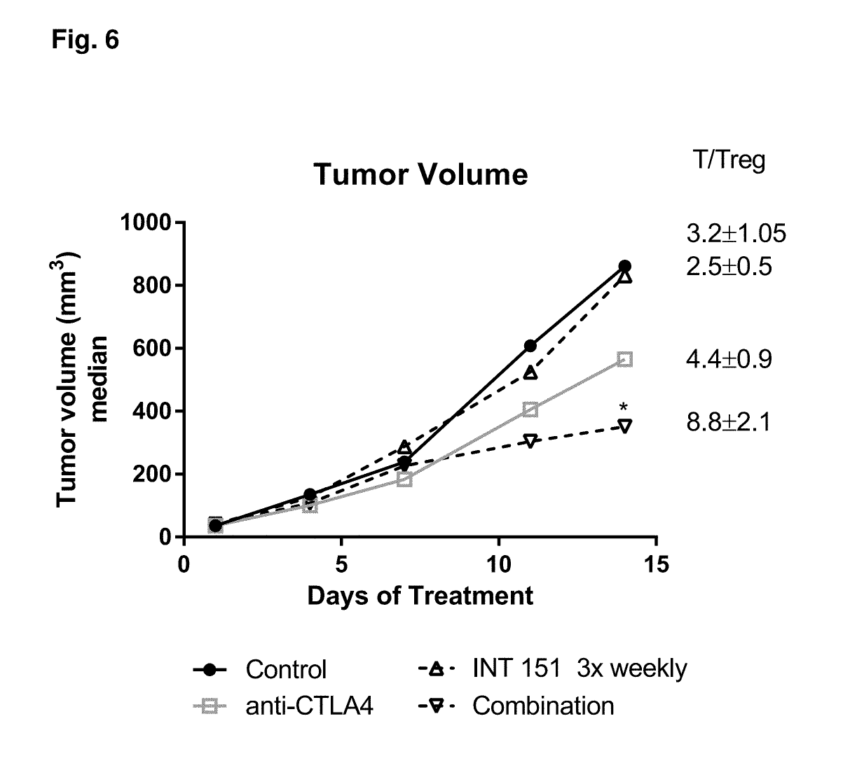

Median tumor volumes were highest in the saline control groups at 861.5 mm³, INT-151 at 831.0 mm³, anti-CTLA-4 measured at 515.5 mm³ and the combination group at 351.0 mm³. Tumor volumes were significantly decreased in mice treated with the combination of anti-CTLA-4 and INT-151 (FIG. 6) compared to the control (p<0.03). The comparison to anti-CTLA-4 treated mice were not significant with p=0.13. Tumor growth inhibition (TGI) showed no effects for INT-151 with a TGI of 4% and for anti-CTLA-4 with a TGI of 34%. TGI for the combination showed moderate efficacy with 60% TGI.

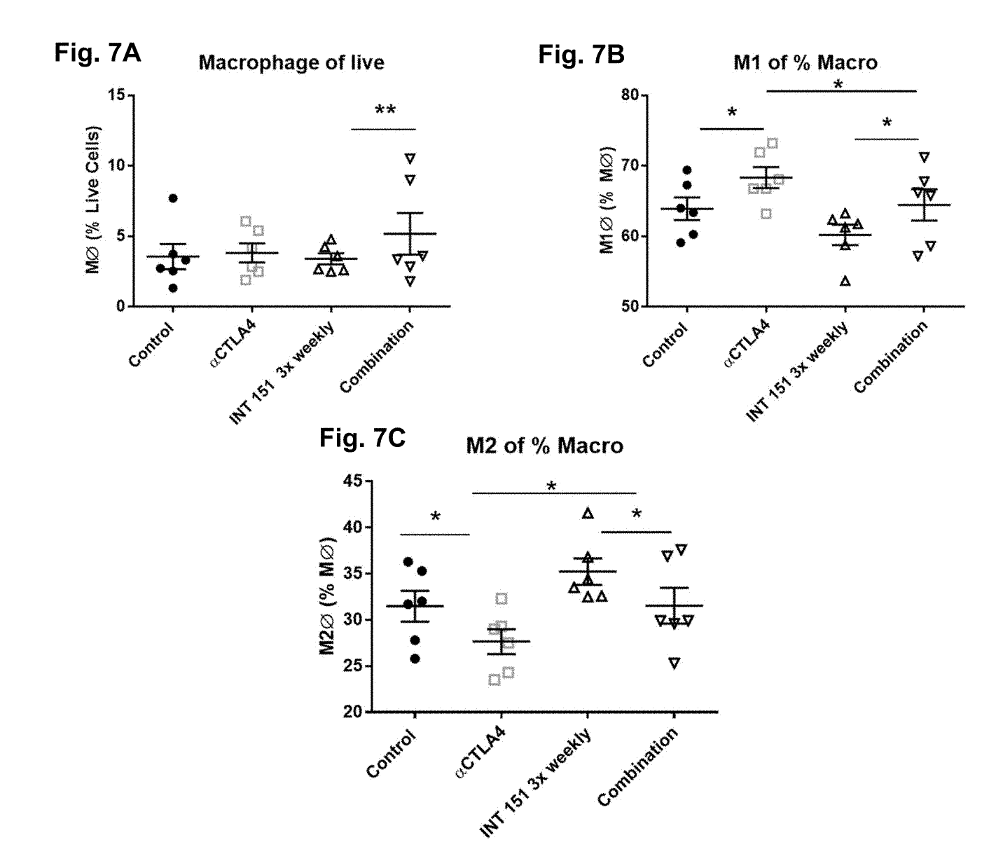

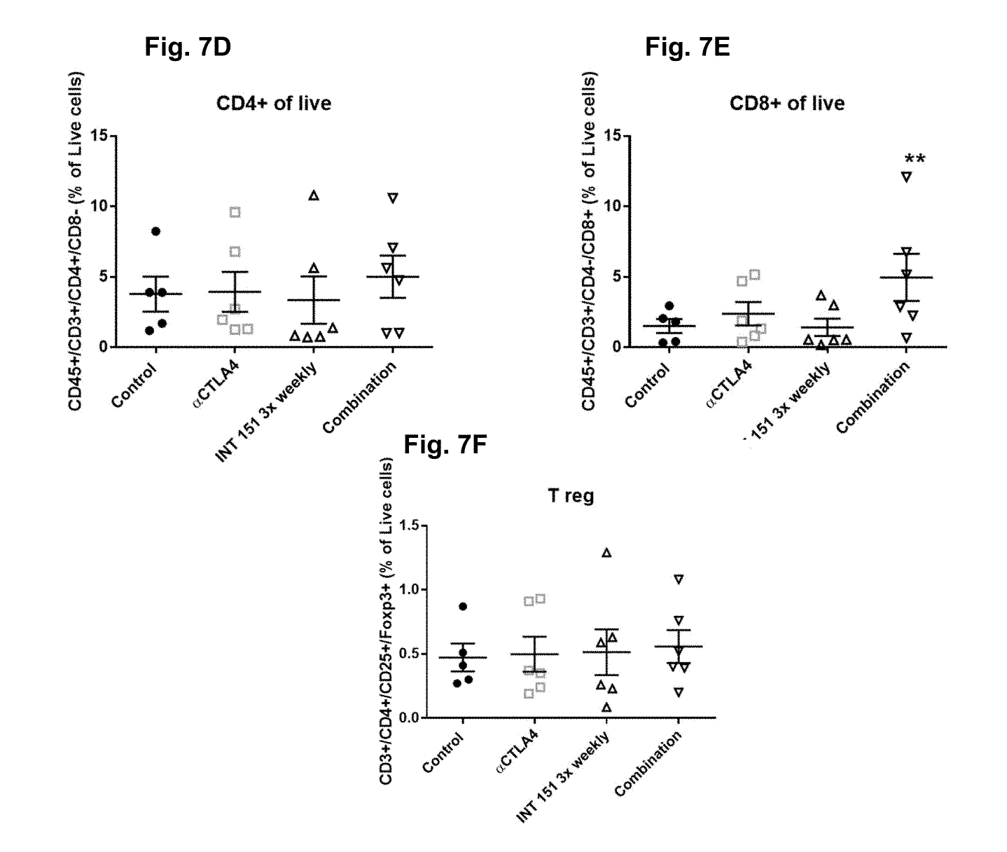

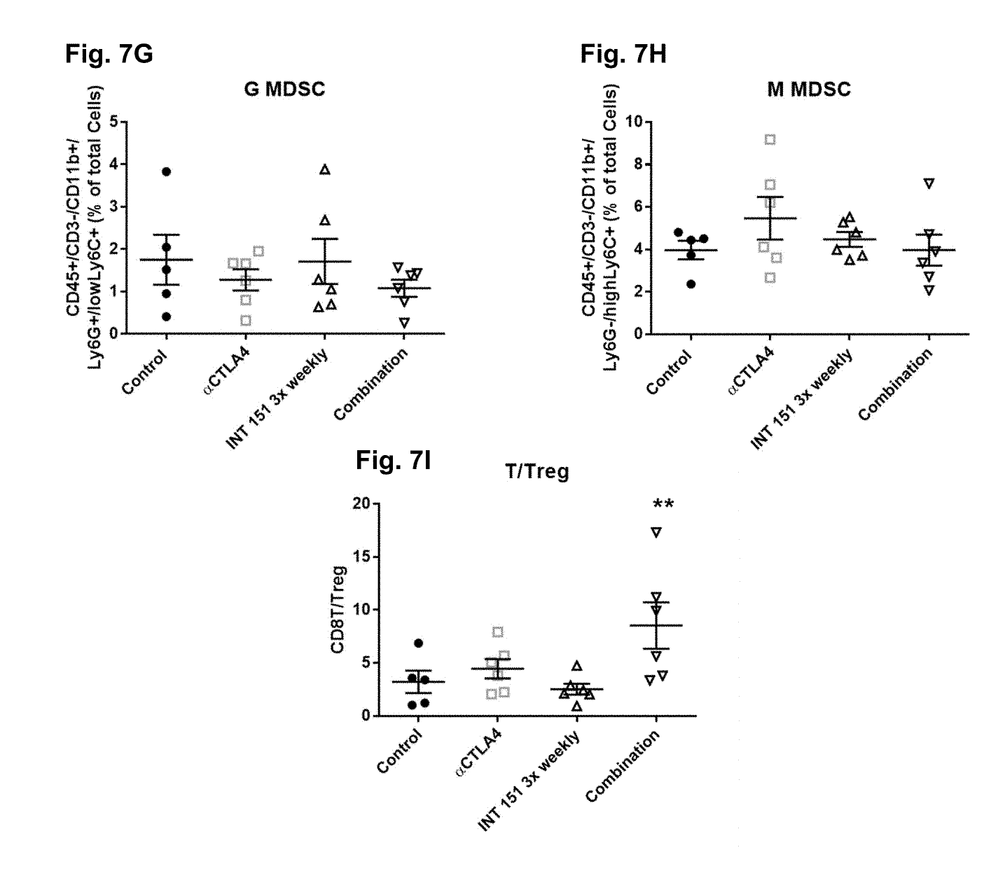

Immune profiles of tumors were assessed using FACS analysis (FIG. 7). INT-151 generated as single agent an anti-inflammatory profile by increasing M2 macrophage population compared to controls, while the M1 macrophage population decreased. INT-151 did not affect regulatory T cell or CD8+ T cell populations. The ratio of CD8+T/Treg was 2.5 and slightly lower than the control group (3.2). CD4+T and granulocytic myeloid suppressor cells were unchanged compared to controls.

Anti-CTLA-4 increased M1 macrophages and decreased M2 macrophages compared to controls (p<0.03). A slight increase of CD8+ T cells was detected that was not significant. Regulatory T cells were not affected. The ratio of CD8+T/Treg was 4.4 and slightly elevated compared to the control group (3.2). Monocytic myeloid derived suppressor cells (MDSC) were slightly increased compared to controls. CD4+T and granulocytic myeloid suppressor cells were unchanged compared to controls.

The combination of INT-151 and α-CTLA-4 increased M1 macrophages (p<0.03). There was no effect on regulatory T cells. CD8+ T cells were increased (p<0.007). The ratio of CD8+T/Treg was the highest of all treatment groups with 8.5 (8.5 vs 3.2, p<0.007 vs control, p<0.0001 vs α-CTLA-4, p<0.01 vs INT-151). CD4+T and granulocytic myeloid suppressor cells were unchanged compared to controls.

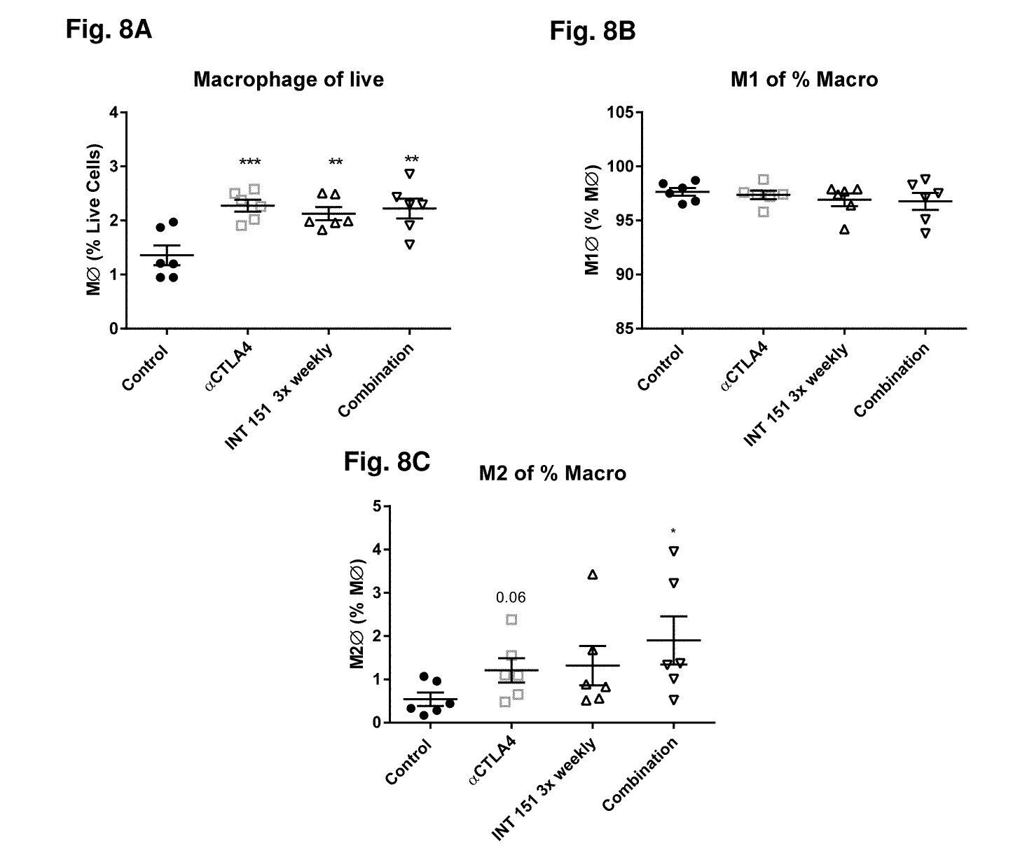

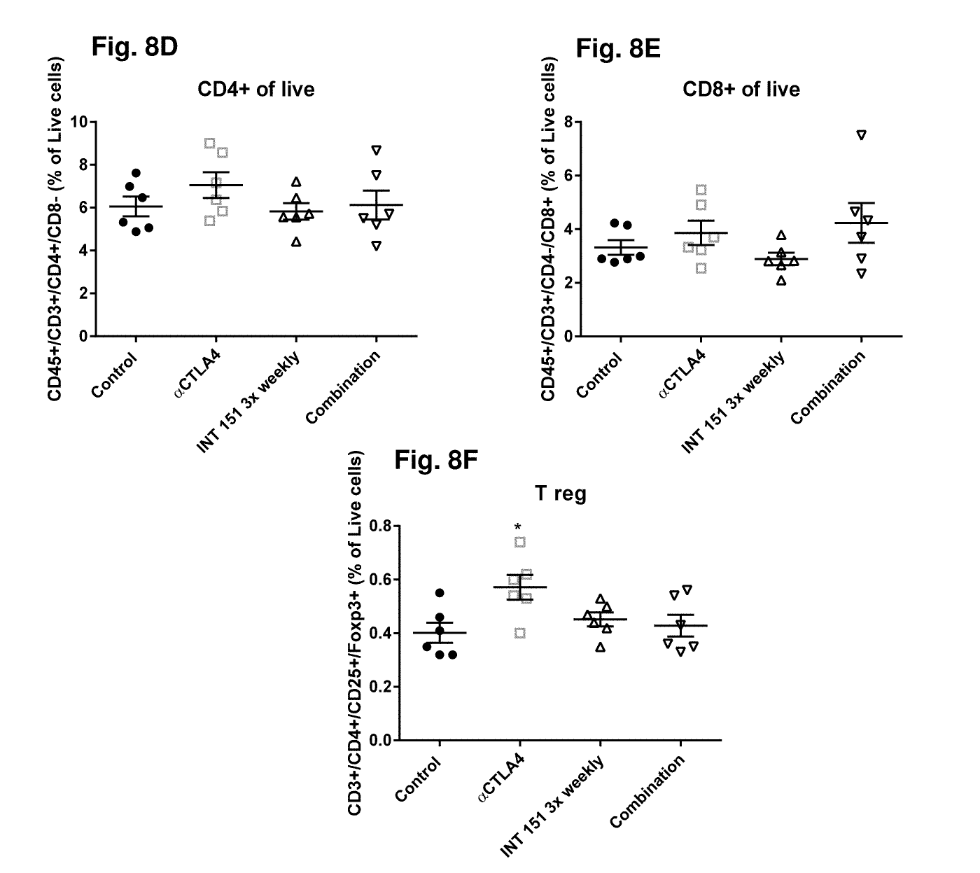

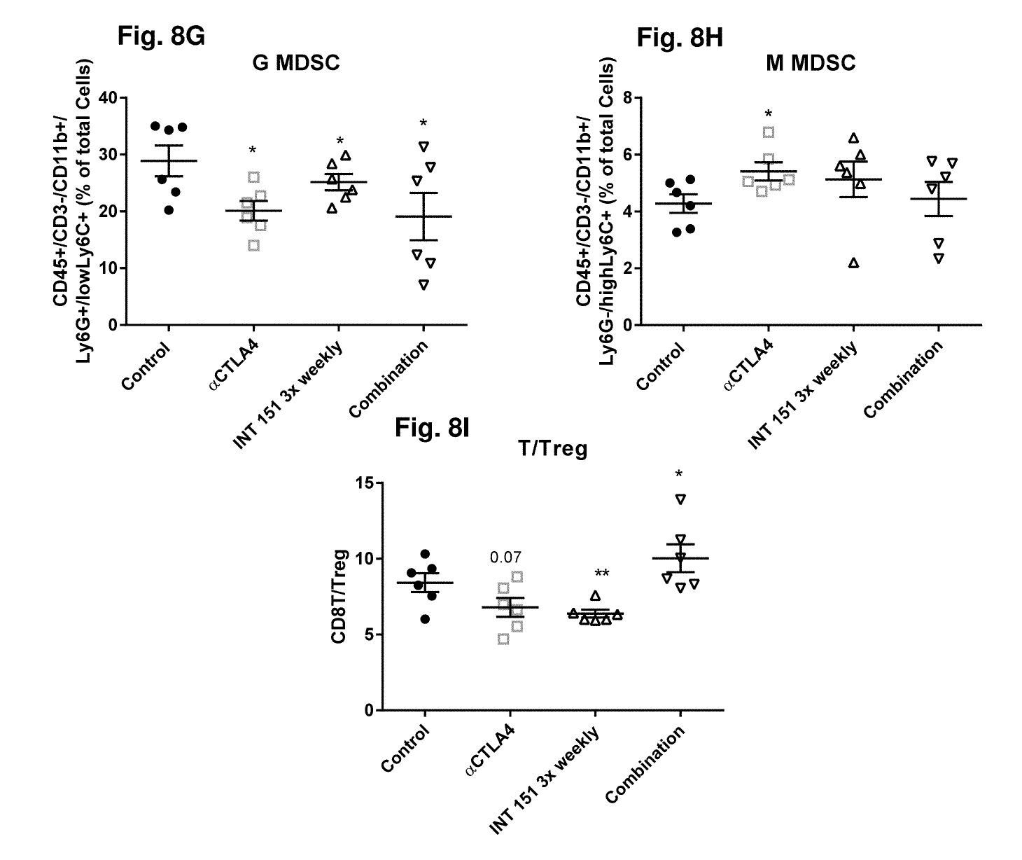

Immune profiles in the blood were assessed using FACS analysis (FIG. 8). INT-151 increased total macrophage population (p<0.001) compared to controls. The M1 macrophage population was unchanged while the M2 macrophage population was elevated compared to controls (p=0.06). INT-151 did not affect regulatory T cell or CD8+ or CD4+ T cell populations. The ratio of CD8+T/Treg was reduced (6.4) compared to controls (8.4) (p<0.05). Monocytic myeloid suppressor cells were not affected while granulocytic MDSCs were reduced compared to controls (p<0.05).

Anti-CTLA-4 increased total macrophage population (p<0.0003) compared to controls. The M1 macrophage population was unchanged while the M2 macrophage population was elevated compared to controls (p=0.06). Anti-CTLA-4 showed increased regulatory T cells (p<0.05) compared to the controls, but did not affect CD8+ or CD4+ T cell populations. The ratio of CD8+T/Treg was reduced (6.8) compared to controls (8.4) but did not reach significance (p=0.07). Monocytic myeloid suppressor cells were increased (p<0.05) while granulocytic MDSCs were reduced compared to controls (p<0.05).

The combination of INT-151 and α-CTLA-4 increased M1 macrophages (p<0.004). There was no effect on regulatory T cells. The ratio of CD8+T/Treg was the highest of all treatment groups with 10.04 (10.04 vs 8.4, p<0.04 vs control, p<0.003 vs α-CTLA-4, p<0.0001 vs INT-151).

Use of INT-151 and its Analogues for T-Cell Expansion Ex Vivo

Expansion of T cells is key to novel approaches in immuno-oncology. CAR-T therapies, which depend on expansion of autologous T-cells and transfection to generate highly specific immune responses, also depend on compounds that increase the expansion of T-cells ex vivo. INT-151 and its analogues can be applied to T cell expansion ex vivo thus adding to an emerging approach to cancer therapy. Previous studies have demonstrated that IMREG-1, containing INT-151 increased T-cell proliferation of leukocytes. (Sinha S. K. et al. (1988) Immunomodulatory components present in IMREG-1, an experimental immunosupportive biologic, Biotechnology 6:810-814; Gottlieb, A. A., et al. (1984) Modulation of human T cell production of migration inhibitory lymphokines by cytokines derived from human leukocyte dialysates, J Immunol 132(1): 256-260.)

Conclusions

The data demonstrated that the combination of anti-CTLA-4 and INT-151 stimulated the immune system on tumor level as well as on blood levels that is favorable for reduction of tumors in this mouse model. The immune profiles confirm the effect on the tumor volumes that were reduced in the combination group.

While this study specifically examined tyrosyl-glycine as a T-cell modifying agent, it is reasonable to expect that all small peptides that have a tyrosyl-glycine as a terminal component would demonstrated similar efficacy and a similar synergistic effect when administered with a checkpoint inhibitor. Specifically, the same synergistic effects observed with a tyr-gly di-peptide would also be expected with Met-Enkephalin or Leu-Enkephalin, for example the penta-peptide tyr-gly-gly-phe-met, tyr-gly-gly-phe-leu, or any derivatives thereof, or pro-peptides thereof.

Similarly, it is reasonable to expect that INT-151 or other like peptides comprising an end-terminal (N-terminal or C-terminal) Tyr-Gly moiety will provide a synergistic anti-cancer effect when administered with any checkpoint inhibitor, given the similar mechanisms of action shared between checkpoint inhibitors that target immune cells (such as lymphocytes, natural killer cells, macrophages and monocytes). Accordingly, it is reasonable to expect that INT-151 will have similar synergistic effects with checkpoint inhibitors that target PD-1 and/or PDL-1.

Based on the data provided herein, it is also reasonable to expect that INT-151 will be synergistic with other anti-cancer immunotherapies that rely on endogenous immune responses, including, but not limited to: T cells directed at cancer using the native T cell receptor (TCR) or genetically modified TCRs, cancer vaccines, and a wide variety of agents that could also partially stimulate the immune system including but not limited to epigenetic modifiers, molecularly targeted therapies, PARP inhibitors, novel necrosis-inducing agents, and radiation therapy.

Combination therapy with INT-151 could yield benefit when given simultaneously, as documented in this study with a checkpoint inhibitor, but also when administered prior to other anti-cancer therapies, or after other anti-cancer therapies.

Further, use of INT-151, since it differs in mechanism of action compared to checkpoint inhibitors, is expected to avoid the adverse events documented by the combination of two different checkpoint inhibitors in the same patient.

Since INT-151 may be administered by a number of routes, including intradermal, subcutaneous, and orally, it is expected that formulations of INT-151 may be equally beneficial when administered by any of these routes.

### Example 2

### A Method for Ex Vivo T Cell Expansion is Described Below

In vitro screening of efficacy for these analogs of INT-151 are conducted with leukocytes isolationed from PBMC preparations as described by Gottlieb et al (Gottlieb, A. A., et al. (1984) Modulation of human T cell production of migration inhibitory lymphokines by cytokines derived from human leukocyte dialysates, J Immunol 132(1): 256-260). PBMC preparations are taken from normal donors and from cancer patients. Positive controls are conducted with met-enkephalin or leu-enkephalin, two opioid peptides known to elicit T cell proliferation in the presence of tetanus toxoid or concanavalin A (Con A) [Sinha S K 1988] or with other cancer specific antigens that may be presented using different approaches, for example Dynabeads coated with CD3/CD28/CD137 or other antigens or antigen presenting dendritic cells (Llobel, F. and Laorden M. L. (1997) Effects of mu-, delta- and kappa-opioid antagonists in atrial preparations from nonfailing and failing human hearts, Gen Pharmacol 28(3): 371-374.)

Because of the importance of opioids in controlling cancer related and surgical pain peptides that produce their immunologic effects independent of the opioid receptor represent more promising candidates as therapeutics. Promising INT-151 analogs, like INT-151, will show activity independent of opioid receptors, which activity is evaluated in experiments where naloxone is added to the preparation to inhibit opioid receptors (Llobel and Laorden 1997). T cell proliferation and activation, a phenotype that favors anti-tumor immunity will be determined by flow cytometry and by interferon gamma release assays. Cytokine assays are conducted to determine additional biological effects, such as IL-2, IL4.

For specific CAR-T cell expansion, methods are optimized based on available procedures for CAR-T cell expansions using cytokine cocktails and various doses of INT-151 peptides or analogues to facilitate T cell expansion.

Ex vivo applications for INT-151 and its analogues may be used to aid antigen specific T cell expansion for novel applications such as CAR-T cell therapies.

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features described herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an amount that is “less than” includes any non-zero amount less than a recited reference number.

Reference to a series of ranges includes ranges which combine the values of the boundaries of different ranges within the series. Thus, to illustrate reference to a series of ranges, for example, of 1-10 and 10-20, includes ranges of 1-20.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The invention is generally described herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless described herein.

The technology illustratively described herein suitably can be practiced in the absence of any element(s) not specifically described herein. Thus, for example, in each instance herein any of the terms “comprising,” “consisting essentially of,” and “consisting of” can be replaced with either of the other two terms. The terms and expressions which have been employed are used as terms of description and not of limitation, and use of such terms and expressions do not exclude any equivalents of the features shown and described or segments thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 80%, at least 85%, at least 90%, at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

The term “specifically binds” refers to a binding agent binding to a target protein or peptide in preference to binding other molecules or other peptides in a particular assay in vitro assay (e.g., an Elisa, Immunoblot, Flow cytometry, and the like). A specific binding interaction discriminates over non-specific binding interactions by about 2-fold or more, often about 10-fold or more, and sometimes about 100-fold or more, 1000-fold or more, 10,000-fold or more, 100,000-fold or more, or 1,000,000-fold or more.

Thus, it should be understood that although the present technology has been specifically disclosed by representative embodiments and optional features, modification and variation of the concepts herein disclosed can be resorted to by those skilled in the art, and such modifications and variations are considered within the scope of this technology.

What is claimed is:

1. A composition comprising (i) an immune-stimulating peptide and (ii) a checkpoint inhibitor.

2. The composition of claim 1, wherein the immune-stimulating peptide comprises the amino acid sequence Tyr-Gly.

3. The composition of claim 1 or 2, wherein the amino acid sequence Tyr-Gly is located on the N-terminal end or C-terminal end of the immune-stimulating peptide.

4. The composition of any one of claims 1 to 3, wherein the immune-stimulating peptide consists of the amino acid sequence Tyr-Gly.

5. The composition of any one of claims 1 to 4, wherein the checkpoint inhibitor comprises an antibody or compound that inhibits, blocks, ameliorates, or suppresses signal transduction through Cytotoxic T-lymphocyte protein 4 (CTLA-4) or Programmed Cell Death 1 (PD-1).

6. The composition of any one of claims 1 to 5, wherein the checkpoint inhibitor comprises an antibody or compound that inhibits, blocks, ameliorates, or suppresses activation of a T-cell, wherein the activation of the T-cell is mediated by signal transduction through CTLA-4 or PD-1.

7. The composition of any one of claims 1 to 6, wherein the checkpoint inhibitor is an antibody or compound that inhibits, blocks, ameliorates, or suppresses binding of CTLA-4 to CD86 (B7-2), CD80 (B7-1) or B7-Related protein (ICOS Ligand, CD275).

8. The composition of any one of claims 1 to 4, wherein the checkpoint inhibitor is an antibody or compound that inhibits, blocks, ameliorates, or suppresses binding of PD-1 to PD-L1 or PD-L2.

9. The composition of any one of claims 1 to 8, wherein the checkpoint inhibitor is a monoclonal antibody, or binding fragment thereof.

9. The composition of any one of claims 1 to 9, wherein the checkpoint inhibitor is a chimeric, humanized or human monoclonal antibody.

10. The composition of any one of claims 1 to 9, wherein the immune-stimulating peptide consists of a dipeptide comprising the amino acid sequence Tyr-Gly, and the checkpoint inhibitor comprises an antibody that binds specifically to CTLA-4 and inhibits, blocks, ameliorates, or suppresses activation of a T-cell upon binding, wherein the activation of the T-cell is mediated by signal transduction through CTLA-4.

11. The composition of any one of claims 1 to 10, wherein the immune-stimulating peptide comprises the formula N-Tyr-Gly-Z or Z-Tyr-Gly-COOH, wherein N is the N-terminal amine of the Tyr, COOH is the carboxy terminus of the Gly and Z is selected from the group consisting of an amino acid, a peptide comprising 2 to 10 amino acids, one or more Tyr-Gly dipeptides, PEG, a PEG mimetic substitute, a sugar, a carbohydrate, a lipid, a nucleic acid, a carrier, a vehicle, an immunogen, one or more bulk amino acids, one or more synthetic amino acids, and combinations thereof.

12. The composition of claim 11, wherein the immune-stimulating peptide comprises an amino acid sequence selected from (i) Tyr-Gly, (ii) Tyr-Gly-Tyr-Gly, (iii) Tyr-Gly-Tyr-Gly-Tyr-Gly, (iv) Tyr-Gly-Tyr-Gly-Tyr-Gly-Tyr-Gly, and (v) Tyr-Gly-Gly-Phe-Met.

13. The composition of claim 11 or 12, wherein the one or more bulk amino acids are selected from Dip (3,3-diphenylalanine), Bip (biphenylalanine), Ath (9-anthracenylalanine), Nal (1-naphthylalanine), Dap (2,3-diaminopropionic acid) and Dab (2,4-diaminobutanoic acid).

14. The composition of any one of claims 11 to 13, wherein the one or more synthetic amino acids comprise a D-amino acid.

15. The composition of any one of claims 11 to 14, wherein the immune-stimulating peptide comprises an amino acid sequence selected from YGGF, YGGDip, YGGBip, YGGAth, YGGDap, YGGDab, YGGNal, and YGGW.

16. The composition of any one of claims 11 to 15, wherein the immune-stimulating peptide comprises an amino acid sequence selected from YGGFGGFG, YGGFGGDipG, YGGFGGBipG, YGGFGGAthG, YGGFGGDapG, YGGFGGDabG, YGGFGGNalG, YGGWGGFG and YGGWGGWG.

17. The composition of any one of claims 11 to 16, wherein the immune-stimulating peptide comprises an amino acid sequence selected from YGGFM, YGGDipM, YGGBipM, YGGAthM, YGGDapM, YGGDabM, YGGNalM and YGGWM.

18. The composition of any one of claims 11 to 17, wherein the immune-stimulating peptide comprises an amino acid sequence selected from YGAF, YGAW, YGAibF, YGAibW, YGAibFM, YGADipM, YGABipM, YGAAthM, YGADapM, YGADabM and YGANalM.

19. A composition comprising an (i) immune-stimulating peptide and (ii) a checkpoint inhibitor wherein the immune-stimulating peptide comprises or consists of an amino acid sequence selected from YAGF, YAGW, YAibGF, YAibGFM. ACYGGGACG, ACYGGFACG, ACYGGBipACG, ACYGGDipACG, ACYGGAthACG, ACYGGDabACG, ACYGGDapACG and ACYGGNalACG.

20. The composition of any one of claims 1 to 19, wherein the compositions is a pharmaceutical composition comprising one or more pharmaceutically acceptable excipients.

21. A method of treating a cancer in a subject in need thereof comprising administering to the subject a therapeutically effective amount of the composition of any one of claims 1 to 20.

22. A method of treating a cancer in a subject in need thereof comprising administering to the subject a therapeutically effective amount of an immune-stimulating peptide and a therapeutically effective amount of a checkpoint inhibitor.

23. The method of claim 21 or 22, wherein the immune-stimulating peptide comprises the amino acid sequence Tyr-Gly.

24. The method of claim 23, wherein the amino acid sequence Tyr-Gly is located on the N-terminal end or C-terminal end of the immune-stimulating peptide.

25. The method of any one of claims 21 to 24, wherein the immune-stimulating peptide consists of the amino acid sequence Tyr-Gly.

26. The method of any one of claims 21 to 25, wherein the checkpoint inhibitor comprises an antibody or compound that inhibits, blocks, ameliorates, or suppresses signal transduction through Cytotoxic T-lymphocyte protein 4 (CTLA-4) or Programmed Cell Death 1 (PD-1).

27. The method of any one of claims 21 to 26, wherein the checkpoint inhibitor comprises an antibody or compound that inhibits, blocks, ameliorates, or suppresses activation of a T-cell, wherein the activation of the T-cell is mediated by signal transduction through CTLA-4 or PD-1.

28. The method of any one of claims 21 to 27, wherein the checkpoint inhibitor is an antibody or compound that inhibits, blocks, ameliorates, or suppresses binding of CTLA-4 to CD86 (B7-2), CD80 (B7-1) or B7-Related protein (ICOS Ligand, CD275).

29. The method of any one of claims 21 to 28, wherein the checkpoint inhibitor is an antibody or compound that inhibits, blocks, ameliorates, or suppresses binding of PD-1 to PD-L1 or PD-L2.

30. The method of any one of claims 21 to 29, wherein the checkpoint inhibitor is a monoclonal antibody, or binding fragment thereof.

31. The method of any one of claims 21 to 30, wherein the checkpoint inhibitor is a chimeric, humanized or human monoclonal antibody.

32. The method of any one of claims 21 to 31, wherein the immune-stimulating peptide consists of a dipeptide comprising the amino acid sequence Tyr-Gly, and the checkpoint inhibitor comprises an antibody that binds specifically to CTLA-4 and inhibits, blocks, ameliorates, or suppresses activation of a T-cell upon binding, wherein the activation of the T-cell is mediated by signal transduction through CTLA-4.

33. The method of any one of claims 21 to 32, wherein the immune-stimulating peptide and the checkpoint inhibitor are administered to the subject at the same time.

34. The method of any one of claims 21 to 32, wherein the immune-stimulating peptide and the checkpoint inhibitor are administered to the subject at different times.

35. The method of claim 34, wherein the immune-stimulating peptide and the checkpoint inhibitor are administered within 5 minutes, within 1 to 12 hours, or within 1 to 2 days of each other.

36. The method of any one of claims 21 to 35, wherein the subject is a human.

37. The method of any one of claims 21 to 36, wherein the cancer is selected from a lymphoma, leukemia, hematopoietic neoplasia, myeloma, melanoma and solid tumor.

38. The method of any one of claims 21 to 37, further comprising administering an anti-cancer vaccine to the subject.

39. The method of any one of claims 21 to 38, further comprising administering a chemotherapeutic agent to the subject.

40. A method of expanding T-cells ex vivo comprising (i) contacting T-cells with an effective amount of the composition of any one of claims 1 to 20, wherein the composition induces mitotic division and/or proliferation of the T-cells.

41. The method of claim 40, wherein the T-cells are obtained from a human subject.

42. The method of claim 41, wherein the T-cells are tumor infiltrating lymphocytes obtained from a tumor.

43. The method of any one of claims 40 to 42, further comprising (ii) contacting the T-cells with a checkpoint inhibitor.

44. The method of any one of claims 40 to 43, further comprising (iii) contacting the T-cells with an antigen presented by a dendritic cell or a carrier.

45. The method of any one of claims 41 to 44, further comprising (iv) re-introducing the T-cells into the human subject after (i), (ii) or (iii).
